(19) United States
(12) Patent Application Publication
(10) Pub. No.: US 2014/0330186 A1
(43) Pub. Date: Nov. 6, 2014

(54) EXTERNAL SENSOR-BASED CONTROL OF ACTIVE TORSO SUPPORT

(71) Applicant: Elwha LLC, Bellevue, WA (US)

(72) Inventors: Roderick A. Hyde, Redmond, WA (US); Jordin T. Kare, Seattle, WA (US); Dennis J. Rivet, Chesapeake, VA (US); Lowell L. Wood, JR., Bellevue, WA (US)

(21) Appl. No.: 13/875,538

(22) Filed: May 2, 2013

(51) Int. Cl. A61F 5/02

(52) U.S. Cl. CPC A61F 5/02
USPC 602/19

(57) ABSTRACT

An active torso support is described. The active torso support may be, for example, an active back brace, and may include one or multiple force applying elements that apply force to localized regions of the torso of a subject. Force applying elements are controlled based upon detection of posture or activity of the subject with a sensor system located at a distance from the active torso support. A signal indicative of posture or activity of the subject is transmitted to the active torso support. In various aspects, the system may include a base station and/or a network of computing devices in communication with the active torso support. Related methods and systems are also described.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None:

### RELATED APPLICATIONS

U.S. patent application Ser. No. 13/721,474, entitled POSTURE DEPENDENT ACTIVE TORSO SUPPORT, naming RODERICK A. HYDE, JORDIN T. KARE, DENNIS J. RIVET, AND LOWELL L. WOOD, JR. as inventors, filed 20 Dec. 2012 with attorney docket no. 1108-004-001-000000, is related to the present application.

U.S. patent application Ser. No. 13/739,868, entitled POSITION SENSING ACTIVE TORSO SUPPORT, naming RODERICK A. HYDE, JORDIN T. KARE, DENNIS J. RIVET, AND LOWELL L. WOOD, JR. as inventors, filed 11 Jan. 2013 with attorney docket no. 1108-004-012-000000, is related to the present application.

U.S. patent application Ser. No. 13/748,871, entitled GAIT-RESPONSIVE ACTIVE TORSO SUPPORT, naming RODERICK A. HYDE, JORDIN T. KARE, DENNIS J. RIVET, AND LOWELL L. WOOD, JR. as inventors, filed 24 Jan. 2013 with attorney docket no. 1108-004-009-000000, is related to the present application.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

To the extent that the listings of applications provided above may be inconsistent with the listings provided via an ADS, it is the intent of the Application to claim priority to all applications listed in the Priority Applications section of either document.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In one aspect, a torso support includes, but is not limited to, at least one force applying element adapted to apply force to a localized region of a torso of a subject; at least one positioning element adapted to position the at least one force applying element with respect to the torso of the subject; at least one receiver adapted to receive at least one activity signal indicative of a posture or activity of the subject detected by at least one sensor system located remote from the torso support; and control circuitry configured to control actuation of the at least one force applying element based at least in part on the at least one activity signal. In addition to the foregoing, other system aspects are described in the claims, drawings, and text forming a part of the disclosure set forth herein.

In one aspect, a torso support system includes, but is not limited to, a remote sensor system including: at least one sensor adapted to detect an input indicative of a posture or activity of a subject; and at least one transmitter adapted for transmitting at least one activity signal indicative of the posture or activity of the subject; and a torso support including: at least one force applying element adapted to apply force to a localized region of a torso of a subject; at least one positioning element adapted to position the at least one force applying element with respect to the torso of the subject; at least one receiver adapted to receive at least one activity signal indicative of the posture or activity of the subject detected by the at least one sensor system located remote from the torso support; and control circuitry configured to control actuation of the at least one force applying element based at least in part on at the least one activity signal received by the at least one receiver. In addition to the foregoing, other system aspects are described in the claims, drawings, and text forming a part of the disclosure set forth herein.

In one aspect, a method of controlling a torso support includes, but is not limited to, receiving at least one activity signal indicative of a posture or activity of a subject wearing a torso support with a receiver on the torso support, wherein the at least one activity signal is indicative of a posture or activity of a subject sensed by a sensor system located remote from the torso support; and controlling actuation of at least one force applying element on the torso support to apply force to a localized region of the torso of the subject based at least in part on the at least one activity signal. In addition to the foregoing, other method aspects are described in the claims, drawings, and text forming a part of the disclosure set forth herein.

In one aspect, an article of manufacture includes, but is not limited to, one or more non-transitory machine-readable data storage media bearing one or more instructions for: receiving at least one activity signal indicative of a posture or activity of a subject wearing a torso support with a receiver on the torso support, wherein the at least one activity signal is indicative of a posture or activity of a subject sensed by a sensor system located remote from the torso support; and controlling actuation of at least one force applying element on the torso support to apply force to a localized region of the torso of the subject based at least in part on the at least one activity signal. In addition to the foregoing, other aspects of articles of manufacture including one or more non-transitory machine readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

For a more complete understanding of embodiments, reference now is made to the following descriptions taken in connection with the accompanying drawings. The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 is an illustration of a torso support system.

FIG. 2 is an illustration of a torso support system in use.

FIG. 3 is a block diagram of a torso support system.

FIG. 4 is an illustration of an embodiment of a torso support system.

FIG. 5A is a block diagram of an embodiment of a torso support system.

FIG. 5B is an illustration of a torso support system of FIG. 5A.

FIG. 6A is a block diagram of an embodiment of a torso support system.

FIG. 6B is an illustration of the torso support system of FIG. 6A.

FIG. 7 is an illustration of an embodiment of a torso support system.

FIG. 8 is a flow diagram of a method of controlling a torso support.

FIG. 9 is a flow diagram of a method of controlling a torso support.

FIG. 10 is a flow diagram of a method of controlling a torso support.

FIG. 11 is a flow diagram of a method of controlling a torso support.

FIG. 12 is a flow diagram of a method of controlling a torso support.

FIG. 13 is a flow diagram of a method of controlling a torso support.

FIG. 14 is a flow diagram of a method of controlling a torso support.

FIG. 15 is a flow diagram of a method of controlling a torso support.

FIG. 16 illustrates an article of manufacture including non-transitory machine-readable data storage media bearing one or more instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

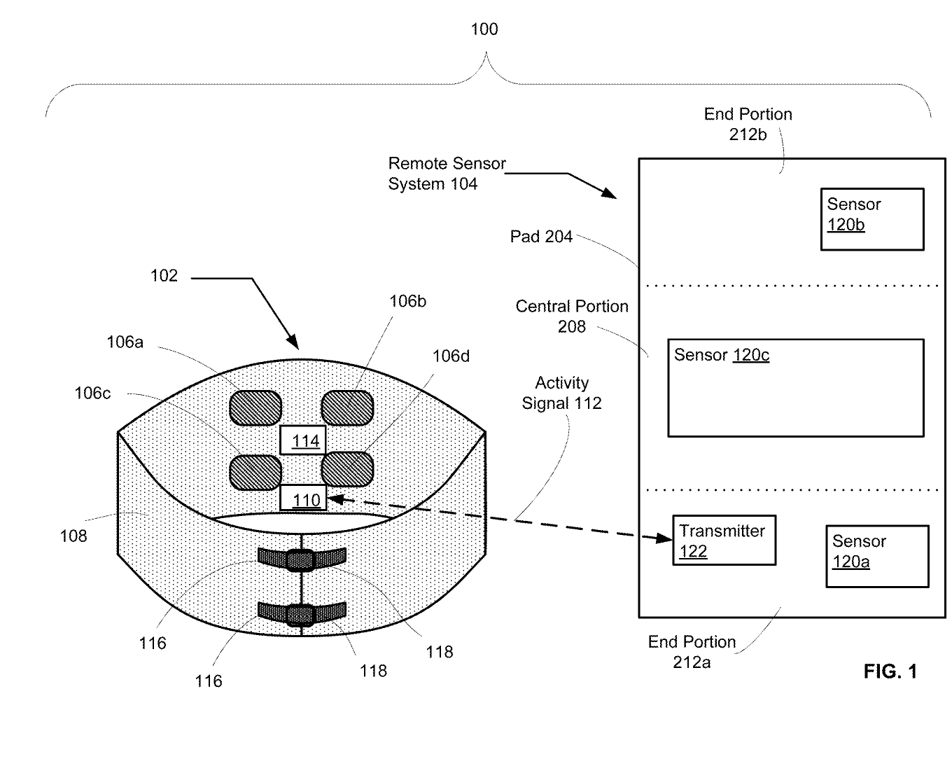

FIG. 1 depicts an embodiment of a torso support system 100 that includes a torso support 102 that is used in combination with a remote sensor system 104. Torso support 102 includes force applying elements 106a-106d, which are adapted to apply force to a localized region of a torso of a subject wearing the torso support; at least one positioning element 108 adapted to position force applying element 106a-106d with respect to the torso of the subject; at least one receiver 110 adapted to receive at least one activity signal 112 indicative of the posture or activity of the subject detected by the at least one remote sensor system 104 located remote from the torso support 102; and control circuitry 114 configured to control actuation of the at least one force applying element 106a-106d based at least in part on at the least one activity signal 112 received by the at least one receiver 110. In some embodiments it may be desirable to provide for two-way communication between torso support 102 and remote sensor system 104, in which case receiver 110 may be a transceiver or other two-way communication device, or a component of communication circuitry that includes one or more transmitter in addition to one or more receiver, and such variations are considered to fall within the scope of the present invention.

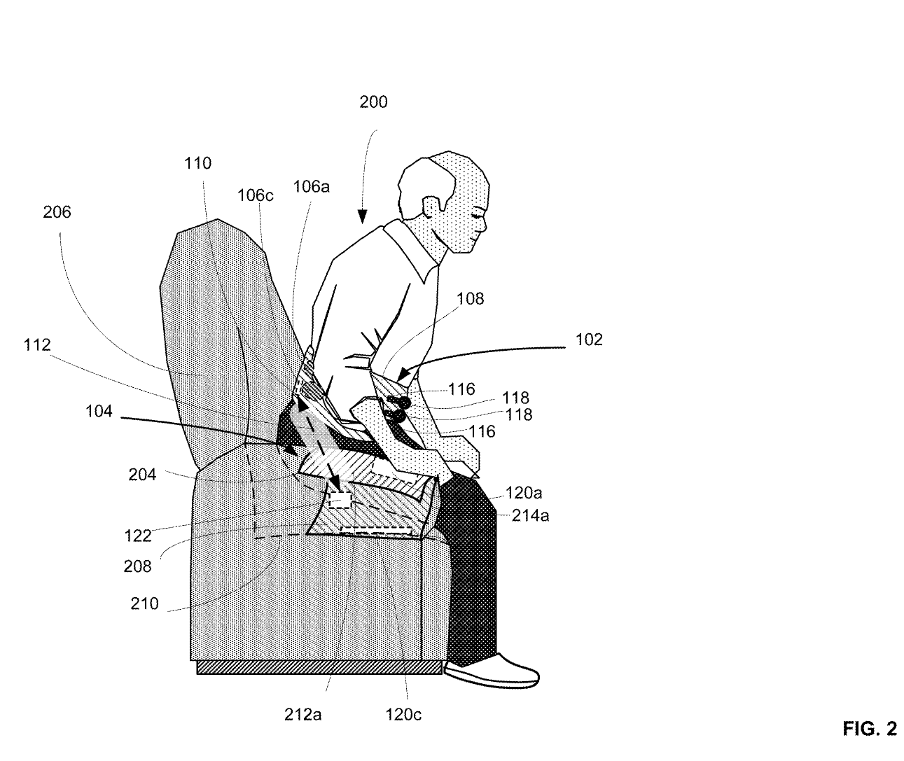

Remote sensor system 104 includes at least one sensor (sensors 120a, 120b, and 120c are shown) used to detect an input indicative of a posture or activity of subject; and at least one transmitter 122 adapted for transmitting activity signal 112, indicative of the posture or activity of the subject. As will be described in greater detail in connection with FIG. 2, remote sensor system 104 is configured as a pad 204 that can be positioned on the seat and armrests of a chair.

Force applying elements 106a-106d can be used to apply force or pressure to a region of the torso of the subject, for example, for the purpose of providing support to weak or injured muscles and/or to prevent or minimize discomfort or injury to muscles or other structures in the torso due to loading. Torso support 102 may be configured as a back support or back brace, as depicted in FIG. 1, but is not limited thereto, and may be configured to support or brace other portions of the torso, including, for example, portions of a back, a side, an abdomen, a chest, a ribcage, a stomach, a hip, a pelvic region, a thoracic region, a shoulder region, a pectoral region, a buttock, a lower back, or an upper back.

It is contemplated that a torso support system as described herein functions generally as follows: if a particular posture or activity of a subject is known to produce motion or loading of muscles and/or bony structures in the subject's torso that is likely to result in injury or discomfort, the active torso support will respond to detection of that posture or activity by the remote sensor system by applying force to one or more appropriate portions of the torso to provide support expected to prevent or minimize injury or discomfort. A person suffering from lower back pain is typically advised to avoid twisting motions, particularly twisting the torso between hips and shoulders, and to reduce the load on back muscles by shifting the load to other muscles, e.g., the arms and legs. Tasks such as lifting, getting in and out of a vehicle or chair, and getting in or out of bed are particularly problematic for a person with back pain. An active torso support as described herein provides additional support to reduce loading, as described herein and illustrated by a number of examples. In situations during which less or no support is needed, force applying elements can be deactivated, or the force applied by the force applying element can be reduced to provide the subject greater freedom of movement, flexibility, or comfort.

In the embodiment depicted in FIG. 1, and in other embodiments described and depicted herein, a force applying element can be any structure that is capable of applying force to a region of the torso of the subject, via a torso-contacting portion such as a pad or probe, and a controllable force-generating component that acts to move the torso contacting portion relative to the torso (e.g. by pressing against the torso and/or by applying shear forces to the torso, e.g. by engaging the surface of the torso by friction). A force applying element can be adapted to fit against a portion of the torso of the subject, where the portion of the torso of the subject is selected from a back, a side, an abdomen, a chest, a ribcage, a stomach, a hip, a pelvic region, a thoracic region, a shoulder region, a pectoral region, a buttock, a lower back, and an upper back. Size, configuration, and force-applying capability of the force applying element are adapted for use with the selected portion of the torso.

Force applying elements (e.g. force applying elements 106a-106d) can be controlled by control circuitry (e.g., control circuitry 114), e.g. via an electrical signal carried via an electrical connection or via a wireless signal such as an optical or electromagnetic signal transmitted from the control circuitry to the force applying element. A force applying element may include one or more actuator, mechanical linkage, expandable element, inflatable element, pneumatic element, hydraulic element, or other structures or components capable of applying force or pressure in a controlled fashion to a localized area of the torso.

A force applying element may be adapted to apply force to the torso of the subject with at least a component of the force in a direction normal to the surface of the torso of the subject. For example, a force applying element can include a plate (which may be curved or planar) a probe, or any structure having shape and size suitable for applying force to a desired portion of the torso. The force applying element may be adapted to apply compressive force to the skin surface.

A force applying element can also include a skin-engaging element adapted to apply tensile or shear force to the skin surface; for example a skin-engaging element may include an adhesive, suction cup, or a frictional surface, or other components known to those skilled in the art to provide for the application of tensile or shear forces to the skin. Thus, a force applying element can be adapted to apply force to the torso of the subject with at least a component of the force in a direction tangential to the surface of the torso of the subject. In an aspect, a force applying element includes a passive force applying element and a controllable active force applying element. In an aspect, a force applying element has a controllable stiffness, a controllable dimension, and/or a controllable position relative to the positioning element. A force applying element can include one or more of a spring, an elastic material, or a viscoelastic material. In an aspect, a force applying element includes an actuator, which may include, for example, a mechanical linkage, an expandable element, an inflatable element, a screw, a pneumatic element, or a hydraulic element. Expandable fluid/air filled bladders, are described, for example, in U.S. Pat. No. 4,135,503 to Romano; U.S. Pat. No. 6,540,707 to Stark et al, and U.S. Pat. No. 5,827,209 to Gross et al., each of which is incorporated herein by reference. Expansion of such bladders can be controlled through the use of a motorized pump and electrically controlled valves, with feedback provided by pressure sensors. Mechanically or pneumatically driven force applying elements can be of the type described in U.S. Pat. No. 5,624,383 to Hazard et al., which is incorporated herein by reference. Pneumatic and hydraulic piston type force applying elements as described in U.S. Pat. No. 6,746,413 to Reinecke et al., which is incorporated herein by reference, and screw thread/worm gear assembly structures as described in U.S. Published Patent Application 2009/0030359 to Wikenheiser et al., which is incorporated herein by reference, may be positioned to press against the torso (delivering force substantially perpendicular to the skin surface), or positioned to apply shear forces (i.e., force having a significant component parallel to the skin surface).

Although positioning element 108 is depicted in FIG. 1 as a belt adapted to be fitted around the waist/mid-torso of a subject, the positioning element can be any structure capable of holding force applying element(s) 106a-106d in position with regard to at least a portion of the torso of the subject, and may include, for example, include at least one band, strap, belt, or harness, or a garment such as a corset, girdle, jacket, vest, or brief. The positioning element may include one or multiple straps or other components, without limitation. The positioning element can be constructed from flexible, resilient, or elastic material, including but not limited to leather, fabric, webbing, mesh, cable, cord, flexible metals or polymers, or sections of rigid metals, polymers or other materials connected in such a manner that the sections can be movably fitted around the torso of the subject, e.g. by a hinge or other linkage or by one or more sections of flexible material. A positioning element (e.g. positioning element 108) may include fasteners to secure the positioning element with respect to the torso of the subject, e.g. straps 116 and buckles 118 as depicted in FIG. 1, or other fasteners as are known in the art, including but not limited to buckles, snaps, zippers, latches, clips, ties, hook and loop fasteners, lacings, and so forth. A positioning element may include an active or passive tensioning component (for example, elastic) to provide for tightening of the positioning element about the torso of the subject to provide for a secure fit. In an embodiment, positioning element may simply include an elastic component which allows it to be slid onto the torso of the subject, without the need for fasteners.

Force applying elements 106a-106d, receiver 110, control circuitry 114, and other system components described herein may be attached to the positioning element 108 or held in place by pressure or friction, e.g. by being pressed between the torso of the subject and the positioning element.

A torso support system as shown in FIG. 1 is depicted in use in FIG. 2. Subject 200 wears torso support 102 around his mid-section, as a back-brace. Positioning element 108 is secured by straps 116 and buckles 118. Force applying elements (e.g. 106a and 106c visible in FIG. 2), also shown in FIG. 1, are positioned against the lower back region of subject 200. Receiver 110 in torso support 102 receives activity signal 112 from transmitter 122 in remote sensor system 104. Remote sensor system 104 is configured as a pad 204 that is placed on the chair 206, with central portion 208 including sensor 120c positioned over seat 210 of chair 206 and end portion 212a including sensor 120a positioned over armrest 214a of chair 206. End portion 212b and sensor 120b of pad 204 are not depicted in FIG. 2, but are as shown in FIG. 1.

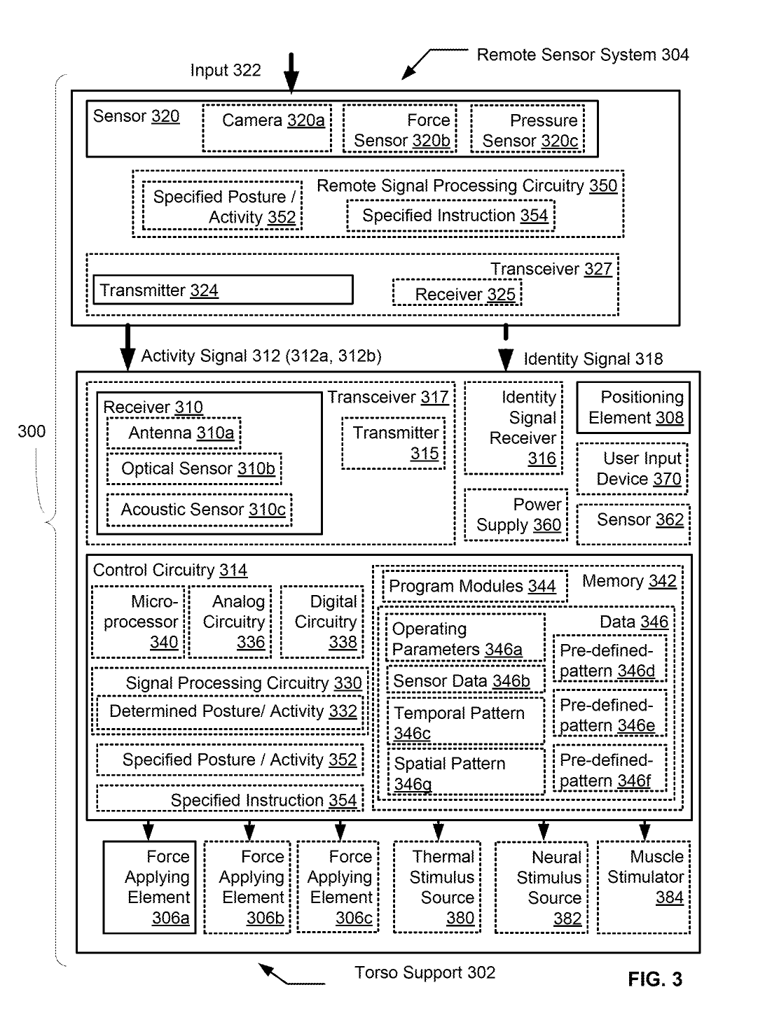

FIG. 3 is a block diagram depicting components of a generalized torso support system 300, which includes torso support 302 and at least one remote sensor system 304. Torso support 302 includes at least one force applying element 306a adapted to apply force to a localized region of a torso of a subject; at least one positioning element 308 adapted to position the at least one force applying element with respect to the torso of the subject; and at least one receiver 310 adapted to receive at least one activity signal 312. Torso support 302 may include additional force applying elements; three force applying elements 306a-306c are depicted in FIG. 3, for the purpose of illustration. However, in some embodiments, only a single force applying element may be used, while in other embodiments, larger numbers of force applying elements may be used. Force applying elements are as described in connection with FIG. 1, and are typically electromechanical in nature. It will be appreciated that a wide range of components may impart mechanical force or motion, such as rigid bodies, spring or torsional bodies, hydraulics, electro-magnetically actuated devices, and/or virtually any combination thereof. As used herein “electro-mechanical system” includes, but is not limited to, electrical circuitry operably coupled with a transducer (e.g., an actuator, a motor, a piezoelectric crystal, a Micro Electro Mechanical System (MEMS), etc). Those skilled in the art will recognize that electro-mechanical as used herein is not necessarily limited to a system that has both electrical and mechanical actuation except as context may dictate otherwise.

Control circuitry 314 is configured to control actuation of the at least one force applying element (e.g. 306a-306c) based at least in part on at the least one activity signal 312 received by the at least one receiver 310. Activity signal 312 is indicative of the posture or activity of the subject detected by the at least one sensor system 304 located remote from the torso support. Remote sensor system 304 includes at least one sensor 320 adapted to detect an input 322 indicative of a posture or activity of a subject; and at least one transmitter 324 adapted for transmitting the at least one activity signal 312 indicative of the posture or activity of the subject. Transmitter 324 can be adapted for transmitting various types of signals, e.g. an electromagnetic signal, a radiofrequency signal, an optical signal, an infrared signal, or an acoustic signal. It will be appreciated that various types of transmitters are known for transmitting the above signals, and the design of a transmitter adapted to transmit one or more type of signal is known to those having skill in the relevant art. See, e.g. U.S. Pat. No. 8,170,656 issued May 1, 2012, to Tan et al., and U.S. Published Application No. 2010/0198067 to Mahfouze et el., dated Aug. 5, 2010, each of which is incorporated herein by reference.

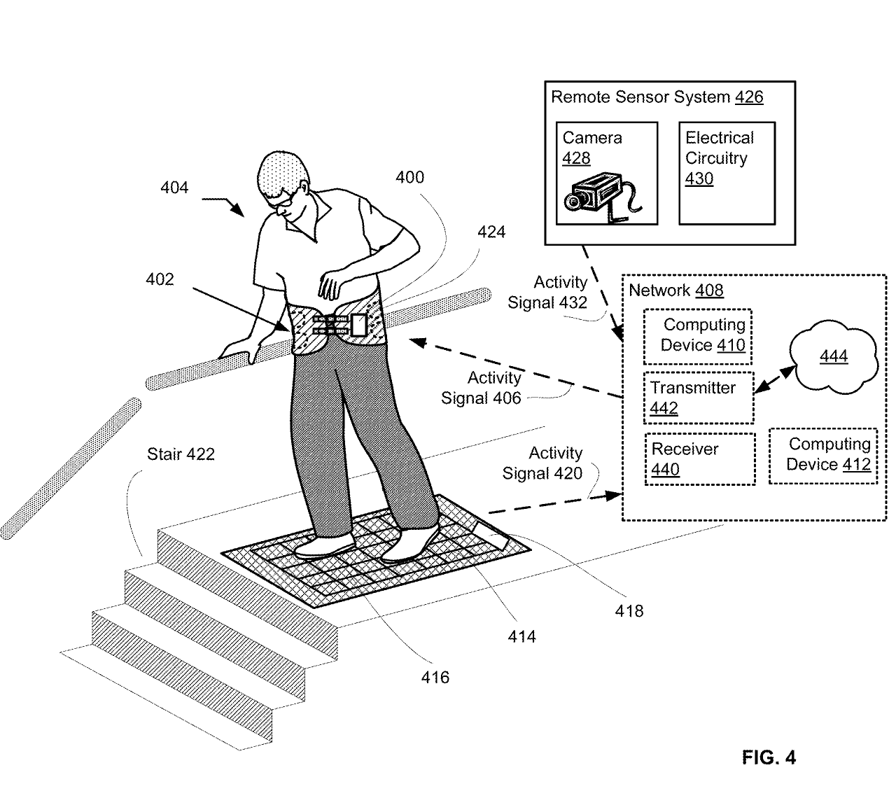

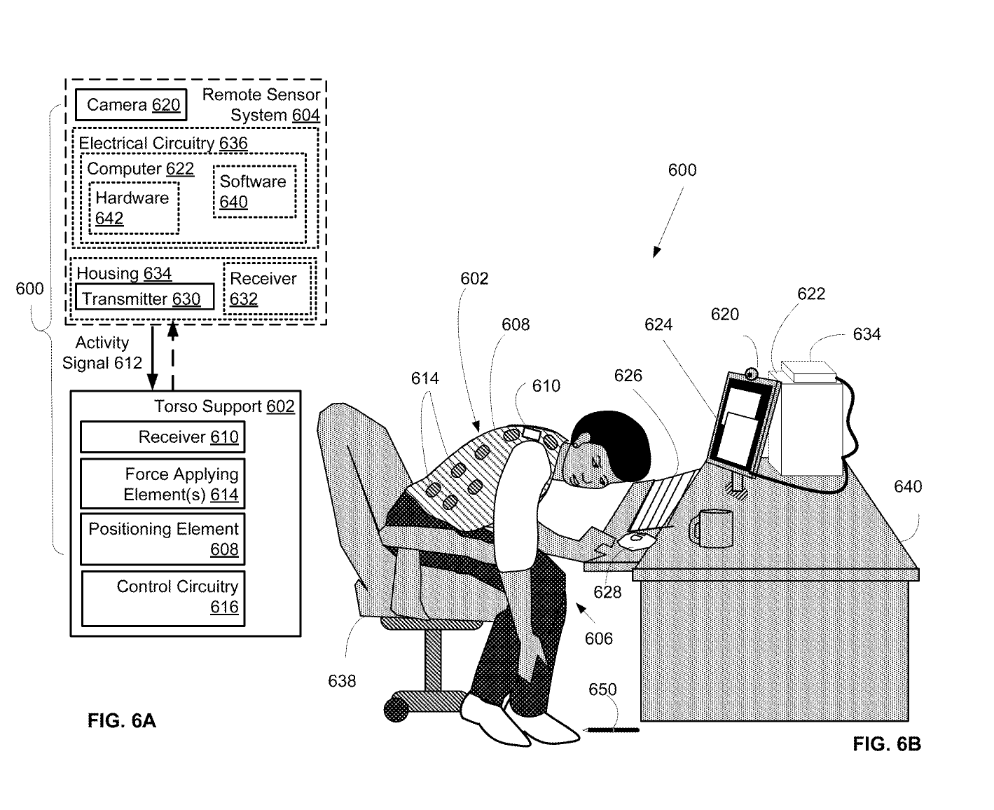

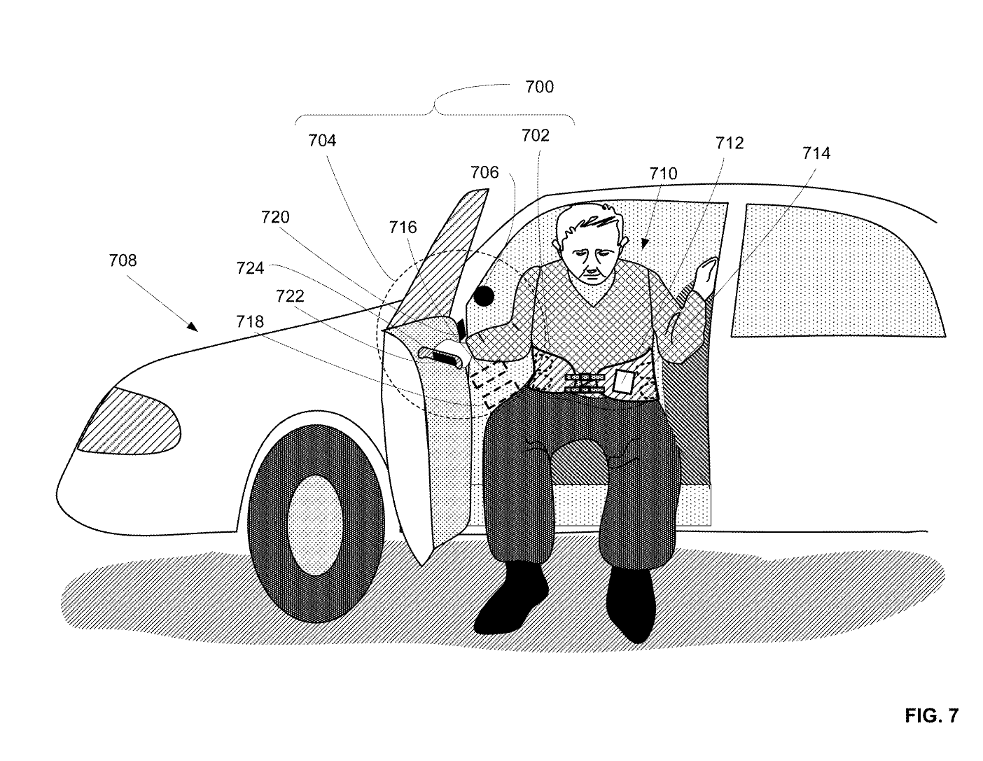

Sensor 320 can include, for example, a camera 320a, a force sensor 320b, a pressure sensor 320c, and/or various other types of sensors as described elsewhere herein or as known to those having skill in the relevant arts. In an aspect, if the at least one sensor in a remote sensor system 304 includes a camera 320a, the camera may be adapted for installation in an environment, as shown in FIG. 4; adapted for use with a computer, as shown in FIG. 6; or adapted for installation in a vehicle, as shown in FIG. 7. Sensor 320 can be a pressure sensor, a motion sensor, a proximity sensor, other types of sensors as known to those having skill in the art. A proximity sensor may be, for example, a micro-impulse radar sensor, an infrared sensor, an optical sensor, an electromagnetic sensor, an acoustic sensor, or any other type of sensor suitable for detecting the proximity of the subject to a location (with the location typically being defined or determined by one or both of the position of the sensor and the source of the signal detected by the sensor.) For example, proximity of the subject can be determined based on the strength of a signal transmitted from the torso support (or other signal source associated with the subject). In another aspect, the proximity of the subject can be determined based on the strength of a signal transmitted from a remote signal source, reflected from the subject, and detected by the sensor. In an aspect, the at least one sensor is adapted for use in connection with a vehicle (e.g., a car as in the embodiment of FIG. 7, or an airplane,), an item of furniture (e.g. a bed or a chair, as in the embodiments of FIGS. 1, 2, 5A and 5B). The at least one sensor may be a component of a seat pad or seat, or a component of a bed or bed pad. In an aspect, the at least one sensor is adapted for use in connection with a floor, e.g. built into the floor, or as a component of a floor mat, as in the embodiment of FIG. 4. In an aspect, the at least one sensor is adapted for use in connection with a door, for example a door in a building, or a door of a vehicle such as a car, truck, or airplane, e.g., as in the embodiment of FIG. 7. In an aspect, the at least one sensor is adapted for use in connection with a hinge, a latch, a door frame, an arm rest, or a handle. The at least one sensor may be adapted for placement in or on a support rail, handle, armrest or handrest. The sensor may be built into any of the above structures during manufacture, or placed on or secured to a support rail, handle, armrest or handrest. For example, one or more sensors may take the form of or be incorporated into a pad, patch, or plate that can be secured to a structure such as a rail, handle, armrest or handrest with adhesive, screws, magnets or other fasteners, or by being placed over the structure and held in place by gravity and/or friction. In an aspect, the at least one sensor may be adapted for use in connection with a stair, for example for detecting that a subject has grasped a stair railing or walked across/through an area leading to or on the stair, e.g. as in the embodiment of FIG. 4.

In an aspect, torso support system 300 may include both receiver 310 and transmitter 315, which in an aspect are components of a transceiver 317, to provide two-way communication with remote sensor system 304 or other system components. Similarly, remote sensor system 304 may include both transmitter 324 and receiver 325, which in an aspect are components of transceiver 327, to provide two-way communication with torso support system 300 or provide for transmission of signals to or receipt of signals from other system components.

In an aspect, receiver 310 in torso support 302 is adapted to receive a wireless signal. The wireless signal may be an electromagnetic signal, e.g., a radio frequency signal, an optical signal, or an infared signal, or it may be an acoustic signal or other wireless signal. Receivers for receiving wireless signals are well known in the electronic arts. For example, receiver 310 may include an antenna 310a suitable for receiving a radio frequency signal, optical sensor 310b for receiving an optical signal or acoustic sensor 310c adapted to receive an acoustic signal.

In an aspect, the at least one transmitter 324 in the remote sensor system 304 is adapted for transmitting at least one activity signal 312 to the at least one receiver 310; in connection therewith, the at least one receiver 310 is adapted to receive the at least one activity signal 312 from the at least one transmitter 324. Such a configuration is depicted, for example, in FIG. 2.

Torso support 302 may also include identity signal receiver 316 adapted to receive a signal 318 indicative of an identity of the subject. Identity signal 318 may be an electromagnetic or optical signal containing or encoding the identity of the subject. In connection therewith, control circuitry 314 is configured to control actuation of the at least one force applying element 306a-306c based at least in part on the identity of the subject. For example, control circuitry 314 may be configured to actuate force applying elements 306a-406c only if the identity of the subject matches the identity of an authorized user. Or, the control circuitry can actuate force applying elements 306a-306c in a particular pattern adapted for a particular subject, based on the identity of the subject. For example, a torso support may be used by different subjects if it is loaned or rented to different subjects by a hospital or other medical equipment supplier. In various aspects, the identity of the subject is determined from an RFID signal, from an identifying number of an electronic device carried by or otherwise associated with the subject (e.g. a cell phone), through facial recognition, or other types of biometric ID. Identity signal receiver 316 may be adapted to receive an electromagnetic signal, optical signal, or acoustic signal, for example.

In an aspect, control circuitry 314 includes signal processing circuitry 330 configured to process the at least one activity signal 312 to determine the posture or activity 332 of the subject. Control circuitry 314 is configured to control actuation of the at least one force applying element (e.g., 306a-306c) based at least in part on the determined posture or activity 352 of the subject.

In various aspects, signal processing circuitry 330 may be configured to process an image signal, a pressure signal, a motion sensor signal, or a proximity sensor signal, for example, to determine the posture or activity of the subject. For example, methods for processing image signals to determine posture and activity are described in U.S. Pat. No. 7,616,779 issued Nov. 10, 2009 to Liau et al., U.S. Pat. No. 8,396,283, issued Mar. 12, 2013 to Iihoshi et al., U.S. Pat. No. 7,330,566, issued Feb. 12, 2008 to Cutler, or U.S. Pat. No. 7,728,839 issued Jun. 1, 2010 to Yang et al., each of which is incorporated herein by reference. In an aspect, signal processing circuitry 330 is configured to process a proximity sensor signal; for example, signal processing circuitry 330 may be configured to determine proximity of the subject to a location based upon a signal strength.

Control circuitry 314 may include analog circuitry 336 or digital circuitry 338. In an aspect, control circuitry 314 may include a microprocessor 340. In an aspect, analog circuitry 336 or digital circuitry 338 are used in combination with microprocessor 340. In an aspect, control circuitry 314 includes software; e.g., control circuitry 314 may include memory 342 or other volatile or non-volatile storage structures to contain program modules 344 used in the operation of torso support 302. Memory 342 may also contain various types of data 346, including but not limited to operating parameters 346a, sensor data 346b, and pattern data 346c, 436d, 346e, and 346f, among others.

Active torso support 302 may include various other elements, including power supply 360, and one or more sensors 362, which may sense various parameters relating to the operation of the torso support or to the status of the subject. For example, sensor 362 may include, for example, an integrating accelerometer or an inclinometer. Data from accelerometers located on the hips of a subject can be used to distinguish walking, turning, ascending or descending stairs, as described in Sabelman et al., (“Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784), which is incorporated herein by reference. Posture sensing may be performed, for example, as described in U.S. patent application Ser. No. 13/721,474, entitled Posture Dependent Active Torso Support, naming Roderick A. Hyde, Jordin T. Kare, Dennis J. Rivet, and Lowell L. Wood, Jr. as inventors, filed 20 Dec. 2012 with attorney docket no. 1108-004-001-000000, which is incorporated herein by reference. Sensor 362 include a motion sensor, for example, as described in U.S. Published Patent Application 2011/0082393, to Bort, dated Apr. 7, 2011, which employs piezoelectric sensors to detect deformation of an orthosis caused by movements of a body region, which is incorporated herein by reference. Other types of sensor 362 include accelerometers, strain gauges, and pressure gauges as described in U.S. Published Patent Application 2001/0020143 to Stark et al., dated Sep. 6, 2001, which is incorporated herein by reference; and force and pressure sensors, as discussed in U.S. Pat. No. 5,827,209 issued Oct. 27, 1998 to Gross, which is incorporated herein by reference.

In an aspect, remote sensor system 304 includes remote signal processing circuitry 350 configured to process the input 322 indicative of a posture or activity of a subject to produce activity signal 312a specifying the posture or activity 352 of the subject. At least one transmitter 324 is adapted for transmitting the activity signal 312a specifying the posture or activity of the subject, and at least one receiver 310 is adapted to receive the at least one activity signal 312a specifying the posture or activity 352 of the subject, detected by the at least one sensor system located remote from the torso support. Control circuitry 314 is configured to control actuation of the at least one force applying element 306a-306c based at least in part on the posture or activity of the subject specified by the at least one activity signal 312a. For example, input 322 may contain information indicative of posture or activity of a signal.

In an aspect, the remote sensor system 304 includes remote signal processing circuitry 350 configured to process the input 322 indicative of a posture or activity of a subject to produce an activity signal 312b specifying least one instruction 354 corresponding to the posture or activity of the subject. At least one transmitter 324 is adapted for transmitting the activity signal 312b specifying the at least one instruction 354, wherein the at least one receiver 310 is adapted to receive the at least one activity signal 312b specifying the least one instruction 354. Control circuitry 314 is configured to control actuation of the at least one force applying element 306a-306c based at least in part on the at least one instruction 354. In another aspect at least one activity signal 312b receivable by the at least one receiver 310 has been processed to specify at least one instruction 354 corresponding to the posture or activity of the subject, wherein the control circuitry 314 is configured to control actuation of the at least one force applying element 306a-306c based on the at least one instruction 354.

As an example of the various alternative signal processing approaches described generally above, input 322 may include, for example, a signal from a pressure sensor on a chair pad or arm rest (e.g. as depicted in FIGS. 1 and 2), which is an analog voltage signal from a pressure sensor 320c that is carried on a wire to transmitter 324. The analog voltage is encoded into an electromagnetic signal (activity signal 312) that is transmitted from transmitter 324 to receiver 310 in torso support system 300, where signal processing may be performed on the signal by signal processing circuitry 330. Alternatively, or in addition, an analog voltage signal from pressure sensor 320c is delivered to remote signal processing circuitry 350, and the signal may be processed to determine a posture or activity specified by the pressure signal. For example, a high pressure signal from a sensor in a chair seat may be indicative of a subject sitting in the chair, whereas a high pressure signal from a sensor in the armrest of a chair may indicate preparation of the subject to sit in the chair. Thus, the specified posture or activity 352 may be, for example “seated” or “preparing to sit.” Accordingly, an activity signal 312a representing particular postures or activities (e.g., activity signal 312a may have a first value to indicate “seated” or a second value to indicate “preparing to sit”) is transmitted from transmitter 324 to receiver 310 in torso support system 300. Control circuitry 314 then controls the torso support based on the specified posture or activity 352 represented in activity signal 312a. As a further alternative, remote signal processing circuitry 350 may process the signal from pressure sensor 320c to specify an instruction 354 corresponding to the posture or activity of the subject. For example, the instruction may be to activate the torso support to provide additional support if the subject is preparing to sit, or deactivating the torso support (or maintaining the torso support in a deactivated state) if the subject is already sitting. Thus activity signal 312b may include a specified instruction (“activate torso support”, “deactivate torso support”) that corresponds to the detected posture or activity of the subject, as determined by remote signal processing circuitry 350.

In an aspect, control circuitry 314 is configured to control actuation of the at least one force applying element 306a-306c based at least in part on a temporal pattern 346c stored in memory 342. Controlling actuation according to a temporal pattern may be as simple as applying a constant force at a selected location for a specific duration (e.g., a duration corresponding to an expected duration of a particular activity), or applying a force that gradually ramps up to a maximum value as a function of time.

In an aspect, control circuitry 314 is configured to initiate actuation of the at least one force applying element 306a-306c based at least in part on the at least one activity signal (312, 312a, or 312b). In another aspect, control circuitry 314 is configured to cease actuation of the at least one force applying element based at least in part on the at least one activity signal (312, 312a, or 312b).

In some aspects, control circuitry 314 is configured to control actuation of the at least one force applying element 306a-306c according to a pre-defined pattern selectable from a plurality of pre-defined patterns, e.g. from pre-defined patterns 346d, 346e, and 346f stored in memory 342. For example, the plurality of pre-defined patterns may include patterns corresponding to a plurality of pre-defined postures or activities of the subject (including, but not limited to, standing, sitting, lying, walking, getting up, sitting down, leaning forward, twisting, or lying down). The pre-defined pattern may be selected from the plurality of pre-defined patterns in a number of ways. For example, the torso support may include a user input device 370, and the pre-defined pattern may be selectable from the plurality of pre-defined patterns based upon an input received by the user input device. Alternatively, or in addition, the pre-defined pattern may be selectable from the plurality of pre-defined patterns based at least in part upon the at least one activity signal (312, 312a, or 312b).

In an aspect, torso support includes at least two spatially separated force applying elements 306a-306c each adapted to apply force to a localized region of the torso of the subject, wherein the at least two spatially separated force applying elements are positioned at different positions with respect to the torso of the subject by the at least one positioning element 308. In an aspect, control circuitry 314 is configured to control actuation of the at least two spatially separated force applying elements based at least in part on a temporal pattern 346c. In addition, or as an alternative, control circuitry 314 is configured to control actuation of the at least two spatially separated force applying elements based at least in part on a spatial pattern 346g. For example, a spatial pattern 346g provides for applying force at several spatially separated locations to support several different muscles (or different portions of a larger muscle) that are loaded or stressed during a particular posture or activity. More complex temporal or spatio-temporal patterns (e.g. cyclical patterns) may also be employed. Control circuitry 314 may be configured to control actuation of the at least two force applying elements 306a-306c according to a pre-defined pattern selectable from a plurality of pre-defined patterns. Again, as discussed above, the torso support may include a user input device 380, and the pre-defined pattern may be selectable from the plurality of pre-defined patterns 346d-346f based upon an input received by the user input device 370. Alternatively, or in addition, the pre-defined pattern may be selectable from the plurality of pre-defined patterns based at least in part upon the at least one activity signal (312, 312a, or 312b). The plurality of pre-defined patterns includes patterns corresponding to a plurality of pre-defined postures or activities of the subject, including one or more of standing, sitting, lying, walking, getting up, sitting down, leaning forward, twisting, or lying down.

In an aspect, control circuitry 314 is configured to control actuation of the at least one force applying element by controlling a pattern of force applied by the at least one force applying element. In another aspect, control circuitry 314 is configured to control actuation of the at least one force applying element by controlling a pattern of motion generated by the at least one force applying element.

In an aspect, active torso support 302 includes thermal stimulus source 380 configured to deliver a thermal stimulus to at least a portion of the torso of the subject. Thermal stimulus source 380 may include, for example, a resistive element, an infrared source, a microwave source, an acoustic energy source, or other elements capable of providing localized heating to the skin or underlying tissues. A thermal stimulus may be applied to stimulate blood circulation, promote healing, enhance comfort of sore or injured muscles, or serve as a counter-stimulus to reduce sensation of pain, for example.

In an aspect, active torso support 302 includes neural stimulus source 382 configured to deliver a stimulus to a neural structure in the torso of the subject. In an aspect, active torso support 302 includes a muscle stimulator 384 configured to deliver a stimulus to a muscle in the torso of the subject. A neural stimulator 382 or muscle stimulator 384 may include an electrode for delivering an electrical stimulus, or one or more coils for delivering a magnetic stimulus, for example, either of which can be driven by an appropriately configured electrical control signal, as known to those having skill in the art. (See, for example, U.S. Pat. No. 8,285,381 issued Oct. 9, 2012 to Fahey et al., which is incorporated herein by reference). Other types of neural or muscle stimulators may be used, as known to those having skill in the art. Nerve and/or muscle stimulation can be used to activate muscles to provide a higher level of strength or stability in the back, or to block or counter pain signals, for example.

As shown in FIG. 3, in an aspect, receiver 310 on a torso support is adapted to receive the at least one activity signal (312, 312a, 312b) from the at least one remote sensor system 304. An example of such a system is depicted in FIGS. 1 and 2.

In another aspect, as shown in FIG. 4, a receiver 400 on a torso support 402 worn by subject 404 is adapted to receive at least one activity signal 406 from a network 408 including one or more computing devices 410, 412 in communication with at least one sensor system 414. In the example of FIG. 4, remote sensor system 414 is a floor mat including resistive sensor grid 416 (as described in Middleton et al., “A floor sensor system for gait recognition,” Fourth IEEE Workshop on Automatic Identification Advanced Technologies, 2005, pp. 171-176, Digital Object Identifier: 10.1109/AUTOID.2005.2, which is incorporated herein by reference). Remote sensor system 414 also includes transmitter 418, configured to transmit an activity signal 420 indicative of the posture or activity of subject 404. In the example of FIG. 4, remote sensor system 414 is located at the top of a stair 422, and is activated when subject 404 walks across it to approach stair 422. For example, force applying elements 424 on torso support 402 may be activated when subject 404 walks across remote sensor system 414, to provide additional support as subject 404 descends stair 422. Torso support 402 may be activated for a fixed amount of time expected to correspond to the amount of time needed to descend stair 422, for example.

In an aspect, the proximity sensor is a perimeter sensor, that is, the proximity sensor is configured to determine whether the subject has crossed a perimeter. Crossing of the perimeter may indicate that the subject is heading toward an area in which activation of the torso support is to be adjusted. For example, a perimeter sensor may be located at the top and/or bottom of a stairway to determine the approach of the subject to the stairway and corresponding need to activate the torso support to provide support to the subject as he or she ascends or descends the stairs. A perimeter sensor may include, for example, an infrared light source positioned on one side of a hallway leading to a stairway and infrared sensor positioned on the opposite side of the hallway such that a person passing through the hallway toward the stairway breaks the beam, producing change in the signal sensed with the infrared sensor. In addition, or as an alternative, network 408 may be in communication with an additional remote sensor system 426. Remote sensor system 426 includes a camera 428 mounted in the environment of subject 404, which includes an area occupied by the subject, which here is depicted as a hallway, but could be, for example a bedroom, an office, a vehicle, a hospital room, a room of a care facility, etc. Electrical circuitry 430 associated with remote sensor system 426 provides for data processing and transmission of activity signal 432 to network 408. The posture or activity of subject 404 can be determined, for example, by image analysis, e.g. as described in U.S. Pat. No. 7,330,566, issued Feb. 12, 2008 to Cutler, or U.S. Pat. No. 7,728,839 issued Jun. 1, 2010 to Yang et al., each of which is incorporated herein by reference. Network 408 includes at least one receiver 440, for receiving activity signal 420 and/or activity signal 432 from remote sensor systems 414 and 426, respectively, and at least one transmitter 442, for transmitting activity signal 406 to receiver 400 on torso support 402. Network 408 includes at least one computing device 410, which is a computing device located locally (for example, in the subject's house), but which may be in communication with other computing devices located either locally (e.g. computing device 412) or remotely, via the internet or other computing network, as represented by “cloud” 444 in FIG. 4. Data acquisition, processing, analysis, and storage may be performed locally or remotely within network 408. In an aspect, the at least one transmitter 418 in remote sensor system 414 is adapted for transmitting at least one activity signal 420 to network 408 including one or more computing devices, and the at least one receiver 400 in torso support 402 is adapted to receive at least one activity signal 406 from network 408. As can be seen, activity signal 406 is not necessarily the same signal as activity signal 420, although it may contain some or all of the same information contained in activity signal 420. Signal 406 may also include additional information, including, e.g. information from activity signal 432.

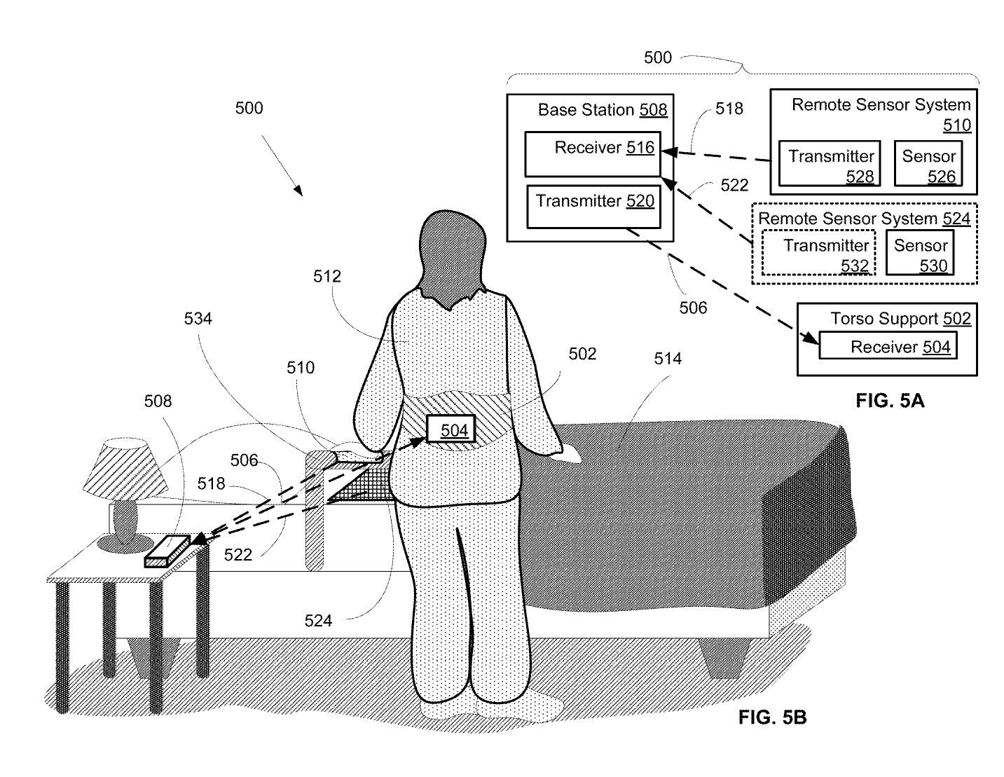

FIG. 5A depicts another example of a torso support system 500 in which a torso support 502 includes a receiver 504 adapted to receive at least one activity signal 506 from a base station 508 which is in communication with at least one sensor system 510. Torso support system 500 is depicted in block diagram form in FIG. 5A, and illustrated in FIG. 5B. Torso support 502 is worn by subject 512 and can be controlled to provide support to the subject's torso as subject 512 gets in and out of a bed 514, for example. In an aspect, base station 508 includes receiver 516 for receiving an activity signal 518 from remote sensor system 510. Base station 508 also includes a transmitter 520 for transmitting an activity signal 506 to receiver 504 in torso support 502. In an aspect, receiver 516 in base station 508 also receives an activity signal 522 from an additional remote sensor system 524. Although base station 508 is depicted as including a single receiver 516 in FIG. 5, in other aspects multiple receivers may be used, without limitation. Similarly, one or multiple receivers may be used in the various embodiments of systems and system components depicted and described herein, as appropriate for the number and types of signals being transmitted between system components. Remote sensor system 510 includes at least one sensor 526 and transmitter 528, and additional remote sensor system 524 includes at least one sensor 530 and transmitter 532. Remote sensor system 510 includes a sensor 526 attached to or incorporated into bed rail 534, and adapted to detect when subject 512 applies pressure to bed rail 534, either in anticipation of getting into bed 514, as depicted in FIG. 5B, or in preparation for rising from bed 514 (not shown). Sensor 526 may detect force, pressure, change in capacitance or conductance, stress, strain, or temperature. In other aspects, sensor 526 is placed on or incorporated into other portions of the bed rail 534 or bed 514. Additional remote sensor system 524, which takes the form of a pad placed on bed 514 (or potentially built into the mattress, bed frame, or other portion of bed 514, can be used to detect whether subject 512 is in bed 514, e.g. by sensing pressure produced by subject 514 lying on a pad. In an aspect, information from remote sensor system 510 and additional remote sensor system 524 can be used to determine whether subject 512 is preparing to get into or out of bed 514. Various other types of sensors could be used to distinguish whether the subject is in the bed or not. Sensed parameters could include, but are not limited to, strain or vibration in the bed frame, or temperature.

FIG. 6A is a block diagram of a torso support system 600, including a torso support 602 and remote sensor system 604, which is configured for use by a subject 606. Torso support system 600 is illustrated in greater detail in FIG. 6B. Torso support 602 includes positioning element 608 configured as a vest worn by a subject 606. Torso support 602 includes a receiver 610 for receiving an activity signal 612, one or more force applying elements 614, and control circuitry 616 for controlling actuation of force applying elements 614.

In remote sensor system 604, the sensor includes a camera 620, which is adapted for use with a computer 622. Camera 620 may be a webcam integrated into monitor 624, or a camera packaged separately and connected to computer 622 via either a wired or wireless connection (for example, a USB 2.0 or 2.0 camera). Such cameras are readily available and well-known in the art. Computer 622 is used in combination with standard input devices such as keyboard 626 and mouse 628, for example. Remote sensor system 604 also includes transmitter 630 which is adapted for transmitting at least one activity signal indicative of the posture or activity of subject 606, sensed by camera 620, and optionally receiver 632. In an aspect, transmitter 630 and receiver 632, if used, may be packaged in housing 634. Housing 634 can be connected to computer 622 and other components of remote sensor system 604 by a wired connection, as depicted in FIG. 6B, or by a wireless connection. As discussed elsewhere herein, receiver 632 may be used to enable two-way communication between remote sensor system 604 and torso support 602. Remote sensor system 604 also includes electrical circuitry 636, which may perform processing of the signal produced by camera 620, for example. The torso support system 600 depicted in FIGS. 6A and 6B is configured for use by subject 606 during the activity of working on computer 622 while seated in chair 638 at desk 640. While computer 622 is suitable for use by subject 606 in a conventional manner (for word processing, computer programming, etc. without limitation), computer 622 may also form a component of remote sensor system 604, and may contain software 640 and/or hardware 642 that form a portion of the electrical circuitry 636 of remote sensor system 604, as shown in FIG. 6A. Alternatively, or in addition, some or all of electrical circuitry 636 may be packaged in housing 634.

Software 640 and hardware 642 can include image processing hardware and/or software used to determine an activity or posture of the subject from an image obtained from camera 620. Such image processing hardware and/or software may, for example, include or generate a model of the background of the image, segment the image, identify the subject in the image, and analyze the image to determine activity or posture of the subject, e.g. based on parameters such as the angle of the torso relative to the hips, or angle of the shoulders relative to the hips. Processing of an image to determine position or posture-related information may be, for example, as described in U.S. Pat. No. 7,616,779 issued Nov. 10, 2009 to Liau et al., U.S. Pat. No. 8,396,283, issued Mar. 12, 2013 to Iihoshi et al., U.S. Pat. No. 7,330,566, issued Feb. 12, 2008 to Cutler, or U.S. Pat. No. 7,728,839 issued Jun. 1, 2010 to Yang et al. If it is determined that the position or posture of subject 606 is one that is expected to result in injury or discomfort (e.g. bending and twisting motion/to retrieve a dropped item such as pencil 650 from the floor is detected through processing of an image obtained with camera 620), force applying elements 614 on torso support 602 are activated in a manner expected to prevent or minimize such injury or discomfort. As described in connection with FIG. 3, activity signal 612 may contain information indicative of the posture or activity of the subject, or in some aspects may specify a posture or activity, or an instruction corresponding to a posture or activity of the subject.

FIG. 7 illustrates an embodiment of torso support system 700, including torso support 702 and remote sensor system 704 in which the sensor includes at least one camera 706 adapted for installation in a car 708. Camera 706 is a driver-facing dashboard camera or similar camera. Torso support 702 is worn by subject 710. Torso support 702 is similar to the torso support described in connection with FIGS. 1 and 2, and includes receiver 712, control circuitry (not shown), and force applying elements 714. Remote sensor system 704 includes remote signal processing circuitry 716 and transmitter 718. In addition, remote sensor system 704 includes door opening sensor 720 and door handle sensor 722.

As noted herein above, getting in and out of a vehicle may be difficult for a person with lower back pain. The person may be advised to break down the motions to separate twisting motion from muscular effort to raise or lower the body. For example, in order to get into a car, subject 710 may be instructed to support the weight of his body with his arms, by holding onto door handle 724, as well as with his legs, and maintain hips and shoulders in alignment while lowering his body into the car seat. Then, after subject 710 is seated in the car seat, he is instructed to swing his legs into car 708 while turning his body, again maintaining hips and shoulders in alignment.

Thus, entry of subject 710 into car 708 is indicated by a signal from door handle sensor 722 produced when subject 710 applies pressure to door handle sensor 722 as a portion of his body weight is transferred to the car via his arm. The process for exiting the car is substantially the reverse; subject 710 swings his legs out of car 708 while turning his body, maintaining hips and shoulders in alignment, and then rises, applying pressure to door handle sensor 722 while using both arm and leg muscles to rise. Entering and exiting the car are preceded by opening of the car door; hence in an aspect, opening of the car door, which is detected by door opening sensor 720, is an indicator of impending activity during which the back will require additional support. Door opening sensors are conventional in modern cars.

While activity on door opening sensor 720 may indicate either that a person is about to enter or exit the car, these two activities can be distinguished by determining whether a person is sitting in the car seat (in which case door opening indicates that the person is about to exit the car) or not (in which case door opening indicates that the person is about to enter the car). Presence of subject 710 in car 708, as well the position/orientation of subject 710, can be detected as described in U.S. Pat. No. 7,396,283 issued Mar. 12, 2013 to Iihoshi et al., which is incorporated herein by reference. Processing of signals from door opening sensor 720, camera 706, and door handle sensor 722, e.g. to determine the posture and activity of subject 710, is performed by remote signal processing circuitry 716, and posture or activity signal is transmitted to receiver 712 in torso support 702 by transmitter 718. Torso support 702 functions as described herein above, to provide support to the back of subject 710 during twisting, sitting, and standing motions that are likely to deleteriously load the subject's back, depending upon the specific needs of subject 710. It will be appreciated that remote signal processing circuitry 716 may be in communication with, or include portions of the electrical circuitry and/or computer system of car 708, in order to receive data from sensors (e.g. door opening sensor 720) built into car 708 and (optionally) to share processing through the use of appropriately configured hardware and software.

In a general sense, those skilled in the art will recognize that the various embodiments described herein can be implemented, individually and/or collectively, by various types of electrical circuitry having a wide range of electrical components such as hardware, software, firmware, and/or virtually any combination thereof, limited to patentable subject matter under 35 U.S.C. §101. Electrical circuitry (e.g. control circuitry 314 and remote signal processing circuitry 350 depicted in FIG. 3, as well as other electrical circuitry) includes electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc), and/or any non-electrical analog thereto, such as optical or other analogs (e.g., graphene based circuitry). In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

Those skilled in the art will recognize that at least a portion of the devices and/or processes described herein can be integrated into a data processing system. Those having skill in the art will recognize that a data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

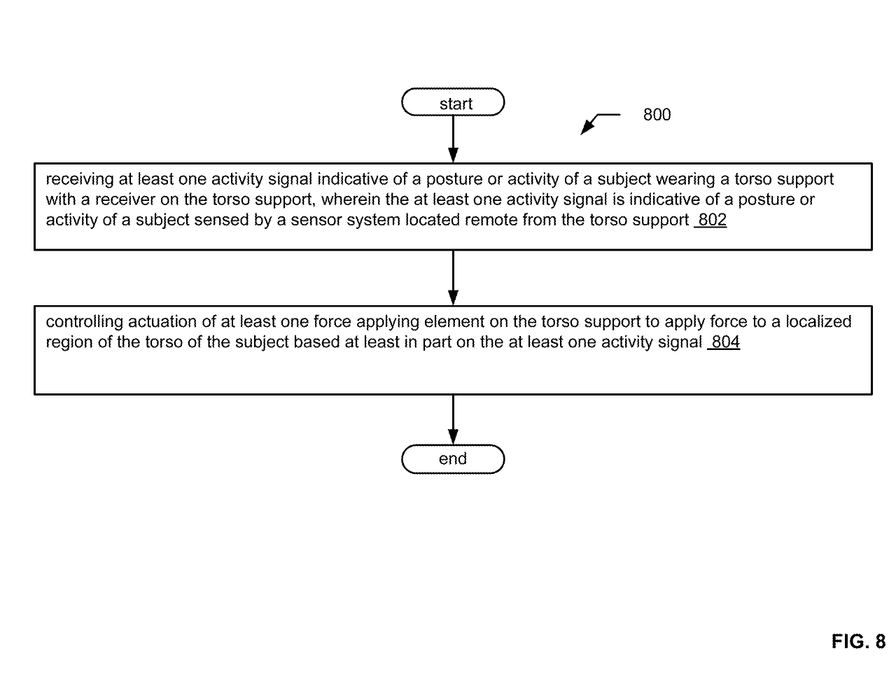

FIG. 8 depicts a method 800 of controlling a torso support. Method 800 includes receiving at least one activity signal indicative of a posture or activity of a subject wearing a torso support with a receiver on the torso support, wherein the at least one activity signal is indicative of a posture or activity of a subject sensed by a sensor system located remote from the torso support at 802; and controlling actuation of at least one force applying element on the torso support to apply force to a localized region of the torso of the subject based at least in part on the at least one activity signal at 804. Here and elsewhere, a solid line around a method step indicates a fundamental aspect of the method, while a dashed line indicates an optional or alternative step that may be included in some, but not all, implementations of the method.

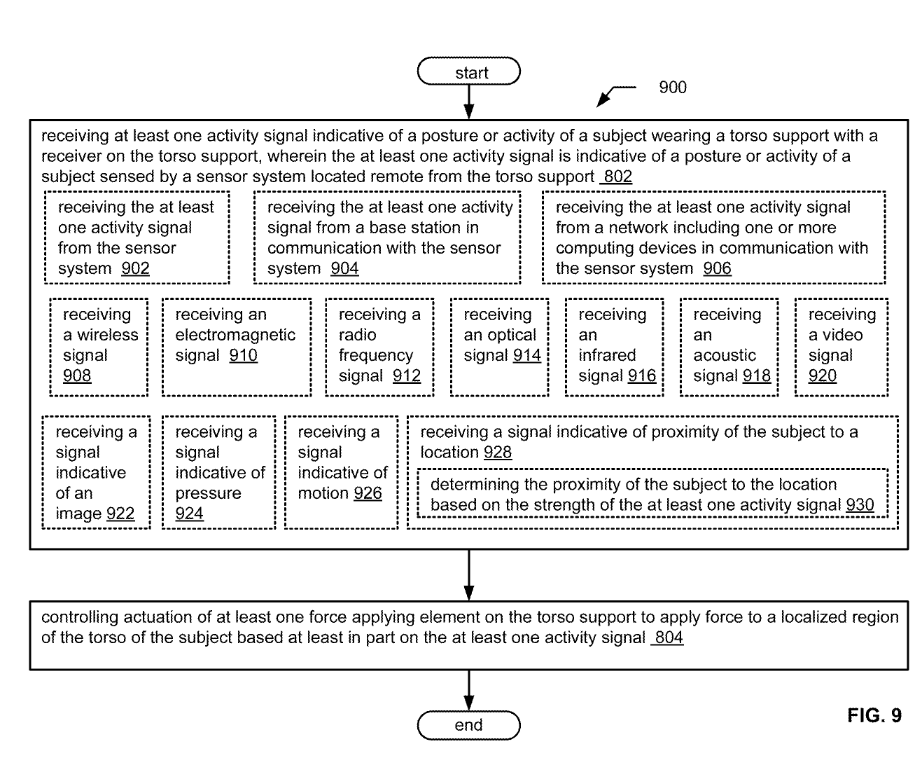

Further method aspects are shown in FIG. 9. Here and elsewhere, steps 802 and 804 are as described in connection with FIG. 8. In an aspect, a method 900 includes receiving the at least one activity signal from the sensor system, as indicated at 902. Such method can be carried out, for example, with a system as depicted in FIG. 2. In another aspect a method includes receiving the at least one activity signal from a base station in communication with the sensor system, as indicated at 904. Such method can be carried out, for example, with a system as depicted in FIG. 5. In another aspect, a method includes receiving the at least one activity signal from a network including one or more computing devices in communication with the sensor system, as indicated at 906. Such method can be carried out, for example, with a system as depicted in FIG. 4. As indicated in FIG. 9, in an aspect receiving the at least one activity signal includes receiving a wireless signal, at 908, which may be, for example, an electromagnetic signal 910, a radio frequency signal 912, an optical signal 914, an infrared signal 916, or an acoustic signal 918. Receiving the at least one activity signal may include receiving a video signal 920 (e.g. from a video camera), a signal indicative of an image 922 (e.g., an image signal, processed image signal, or other signal containing image data or other representation of an image), a signal indicative of pressure 924, motion 926, or proximity of the subject to a location 928. For example, the method can include determining the proximity of the subject to the location based on the strength of the at least one activity signal, as indicated at 930.

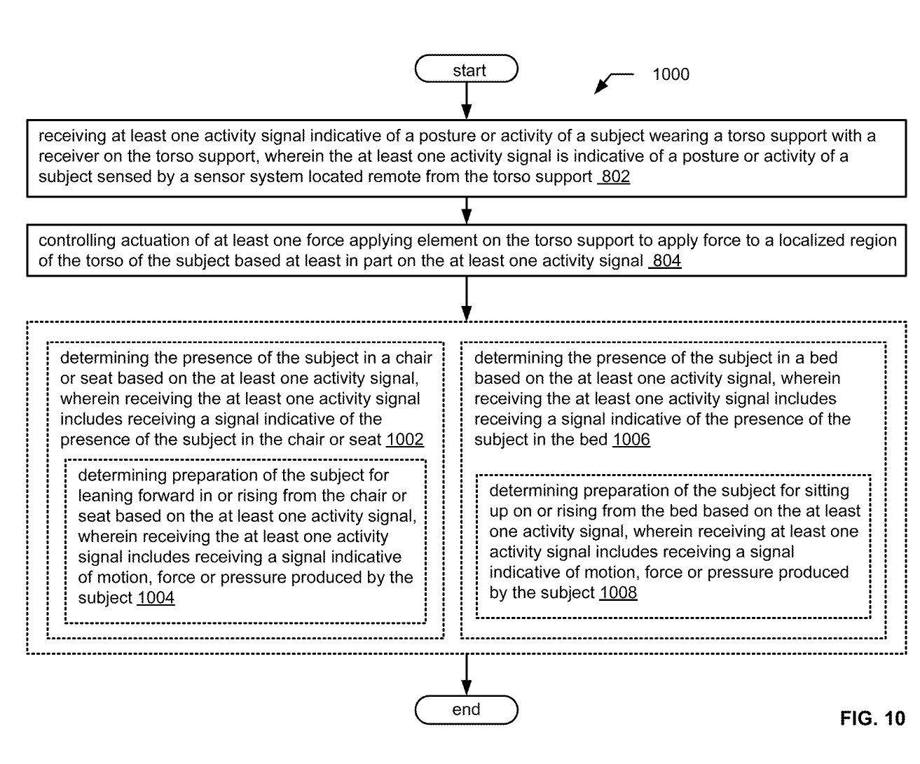

FIG. 10 depicts further aspects of the method of FIG. 8. In an aspect, a method 1000 includes determining the presence of the subject in a chair or seat based on the at least one activity signal, wherein receiving the at least one activity signal includes receiving a signal indicative of the presence of the subject in the chair or seat, as indicated at 1002. In various aspects, a signal from a pressure sensor, force sensor, temperature sensor, motion sensor, or vibration sensor, located in or on a portion of the chair or seat could be used to detect whether the subject was in the chair or seat. For example, sensor 120c in FIGS. 1 and 2 produces a signal indicative of the presence of the subject in chair 206. Method 1000 may also include determining preparation of the subject for leaning forward in or rising from the chair or seat based on the at least one activity signal, wherein receiving the at least one activity signal includes receiving a signal indicative of motion, force or pressure produced by the subject, at 1004. Referring again to the embodiment depicted in FIGS. 1 and 2, sensors 120a and 120b on the arms of chair 206 can be activated when the subject presses down on them, indicating preparation of the subject to rise from chair 206. In other aspects, in the system depicted in FIG. 6, camera 620 can be used to obtain an image of subject 606 that can be analyzed to determine whether subject 606 is present in chair 638, and/or whether subject 606 is leaning forward or making other movements that indicate preparation to rise from chair 638.

In another aspect, method 1000 as shown in FIG. 10 includes determining the presence of the subject in a bed based on the at least one activity signal as indicated at 1006, wherein receiving the at least one activity signal includes receiving a signal indicative of the presence of the subject in the bed. For example, a signal from pressure sensor, force sensor, temperature sensor, motion sensor, or vibration sensor, located in or on a portion of the bed could be used to detect whether the subject was in the bed. In addition, method 1000 may include determining preparation of the subject for sitting up on or rising from the bed based on the at least one activity signal, wherein receiving at least one activity signal includes receiving a signal indicative of motion, force or pressure produced by the subject, as indicated at 1008. For example, when the subject wishes to sit up or rise from the bed, the subject may grasp a bed rail 534 (as depicted in FIG. 5), or place his or her hand(s) on the edge of the bed to help support his or her body and reduce strain on his or her back. Therefore, preparation of the subject for sitting up or rising may be indicated by a signal from force, pressure sensor, motion sensor, or vibration sensor, located in or on a bedrail, headboard, edge of the bed, or other portion of the bed that is loaded by the subject in preparation for and/or during sitting up in or rising from the bed.

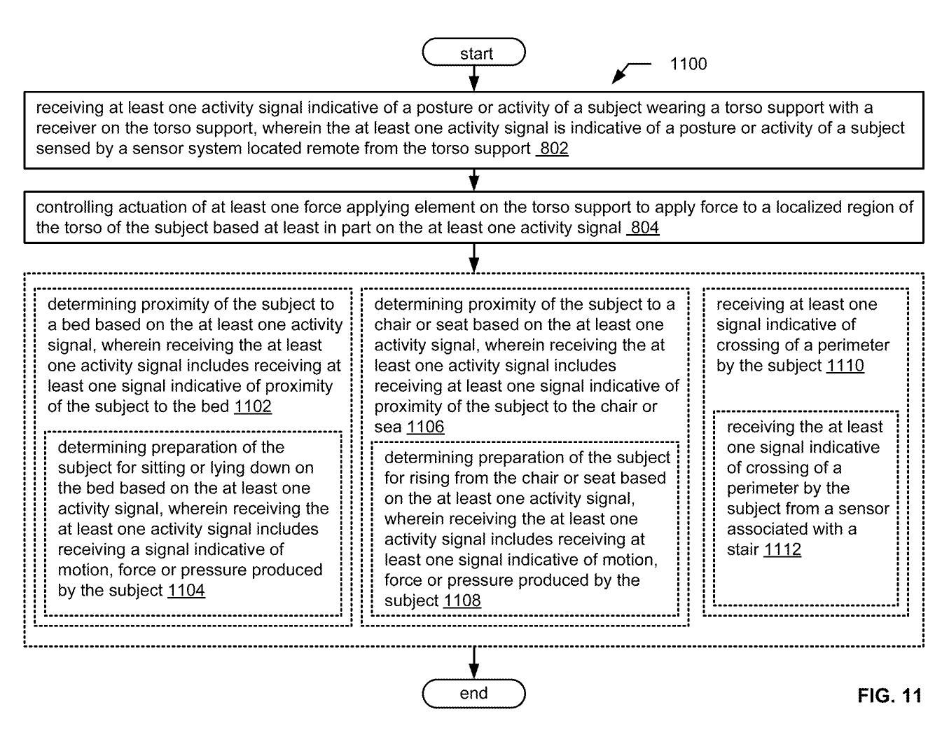

A shown in FIG. 11, in an aspect a method 1100 includes determining proximity of the subject to a bed based on the at least one activity signal, wherein receiving the at least one activity signal includes receiving at least one signal indicative of proximity of the subject to the bed, as indicated at 1102. In addition, the method includes determining preparation of the subject for sitting or lying down on the bed based on the at least one activity signal, wherein receiving the at least one activity signal includes receiving a signal indicative of motion, force or pressure produced by the subject at 1104.

Alternatively, or in addition, method 1100 includes determining proximity of the subject to a chair or seat based on the at least one activity signal, wherein receiving the at least one activity signal includes receiving at least one signal indicative of proximity of the subject to the chair or seat, as indicated at 1106. In addition, method 1100 may include determining preparation of the subject for rising from the chair or seat based on the at least one activity signal, wherein receiving the at least one activity signal includes receiving at least one signal indicative of motion, force or pressure produced by the subject, as indicated at 1108.

In another aspect, receiving the at least one activity signal includes receiving at least one signal indicative of crossing of a perimeter by the subject, as indicated at 1110. For example, method 1100 may include receiving the at least one signal indicative of crossing of a perimeter by the subject from a sensor associated with a stair 1112, e.g. as depicted in FIG. 4.

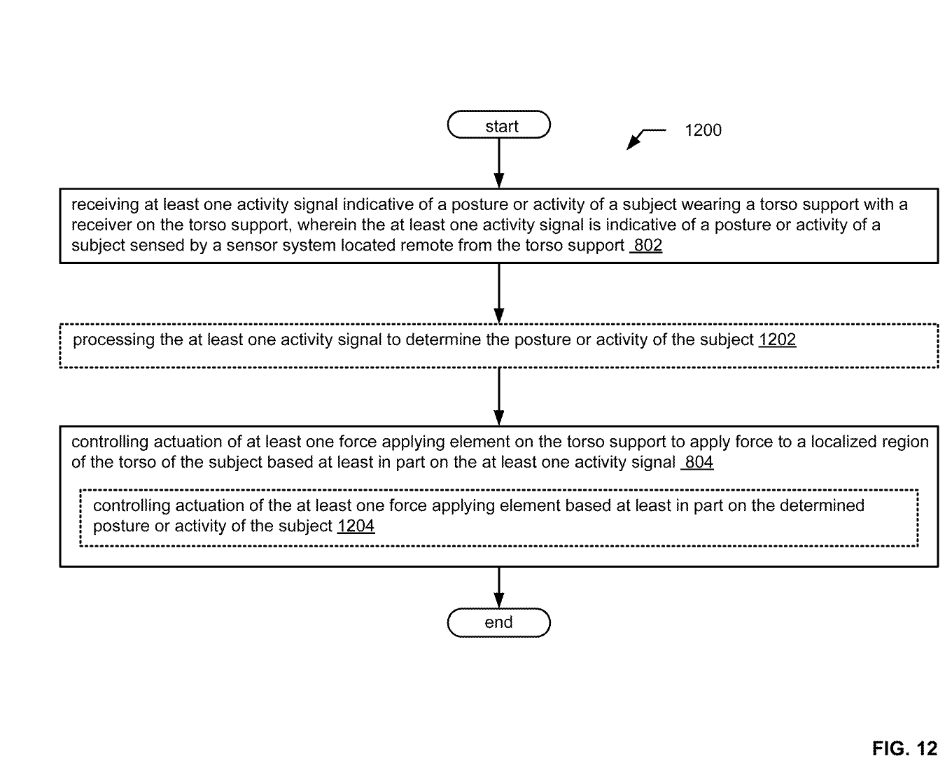

As shown in FIG. 12, in an aspect, a method 1200 includes processing the at least one activity signal to determine the posture or activity of the subject, at 1202. Method 1200 can then include controlling actuation of the at least one force applying element based at least in part on the determined posture or activity of the subject, as indicated at 1204. Determined posture or activity can be obtained as described in connection with FIG. 3, for example.

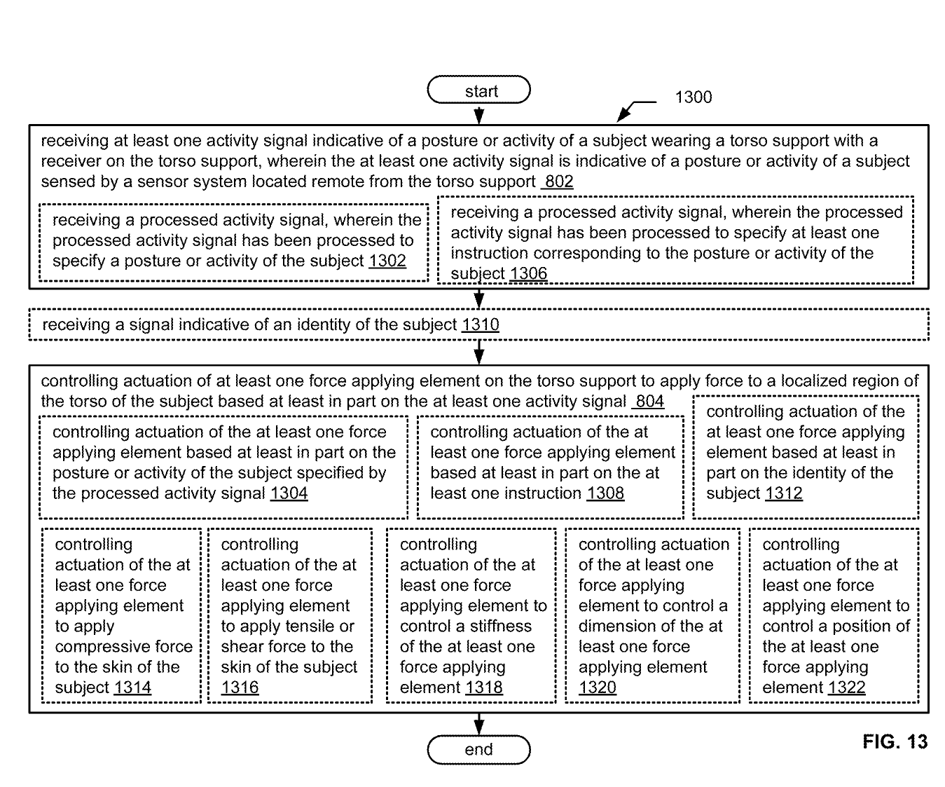

In another aspect, as shown in FIG. 13, in related method 1300, receiving the at least one activity signal includes receiving a processed activity signal, wherein the processed activity signal has been processed to specify a posture or activity of the subject, as indicated at 1302. In connection therewith, method 1300 includes controlling actuation of the at least one force applying element based at least in part on the posture or activity of the subject specified by the processed activity signal, as indicated at 1304.

In another aspect of method 1300, receiving the at least one activity signal includes receiving a processed activity signal, wherein the processed activity signal has been processed to specify at least one instruction corresponding to the posture or activity of the subject, as indicated at 1306. In connection therewith, method includes controlling actuation of the at least one force applying element based at least in part on the at least one instruction, as indicated at 1308. The use of a processed activity signal that specifies the posture or activity of the subject, or an instruction corresponding to the posture or activity of the subject, is described in connection with FIG. 3.

In yet another aspect, method 1300 includes receiving a signal indicative of an identity of the subject, at 1310, and subsequently controlling actuation of the at least one force applying element based at least in part on the identity of the subject, at 1312, as described in connection with FIG. 3.

In various aspects, method 1300 includes controlling actuation of the at least one force applying element to apply compressive force to the skin of the subject, at 1314, apply tensile or shear force to the skin of the subject, at 1316, control a stiffness of the at least one force applying element, at 1318, control a dimension of the at least one force applying element, at 1320, or control a position of the at least one force applying element, at 1322.

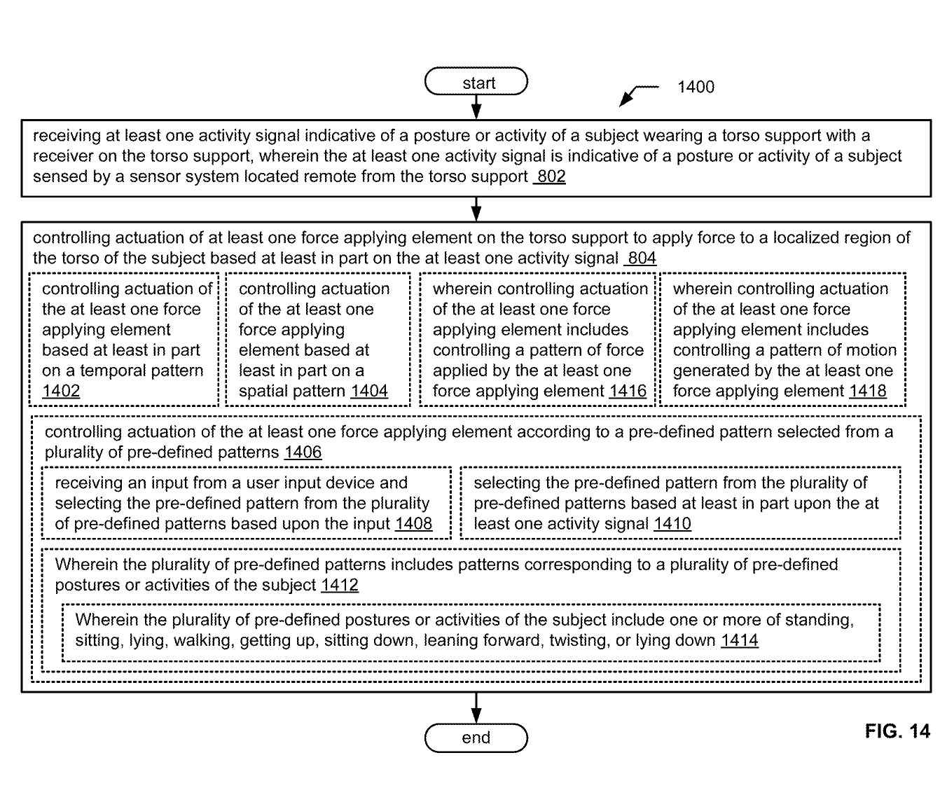

FIG. 14 illustrates a method 1400 including aspects relating to controlling actuation of the at least one force applying element according to patterns. In an aspect, method 1400 includes controlling actuation of the at least one force applying element based at least in part on a temporal pattern, as indicated at 1402. In an aspect, method 1400 includes controlling actuation of the at least one force applying element based at least in part on a spatial pattern, at 1404. It will be appreciated that force and motion may be related, depending upon the mechanical properties of the torso support and the portion of the body of the subject to which force is applied, but that either force or motion (or a parameter derived therefrom) may be measured and used as a control parameter.

In an aspect, method 1400 includes controlling actuation of the at least one force applying element according to a pre-defined pattern selected from a plurality of pre-defined patterns, as indicated at 1406. Method 1400 may include receiving an input from a user input device and selecting the pre-defined pattern from the plurality of pre-defined patterns based upon the input, as indicated at 1408, selecting the pre-defined pattern from the plurality of pre-defined patterns based at least in part upon the at least one activity signal, as indicated at 1410, or a combination thereof. The plurality of pre-defined patterns may include patterns corresponding to a plurality of pre-defined postures or activities of the subject, as indicated at 1412, which may include, for example, one or more of standing, sitting, lying, walking, getting up, sitting down, leaning forward, twisting, or lying down, as indicated at 1414.

Controlling actuation of the at least one force applying element can include controlling a pattern of force applied by the at least one force applying element, as indicated at 1416, or controlling a pattern of motion generated by the at least one force applying element, as indicated at 1418.

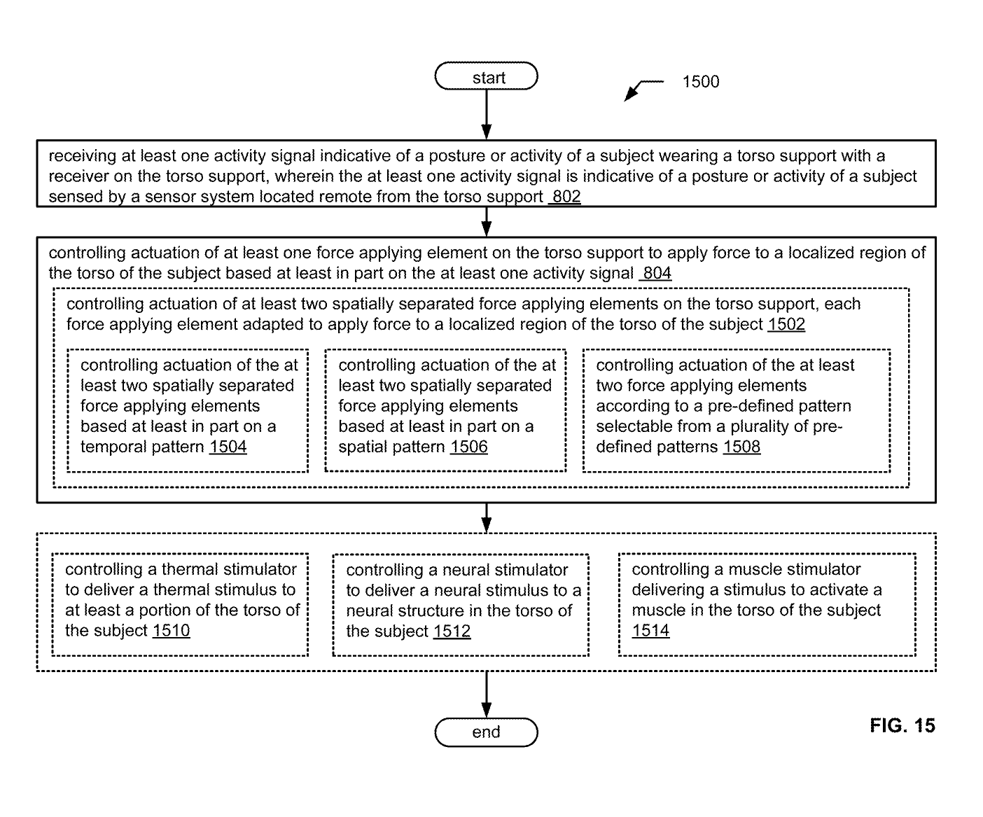

In another aspect, as shown in FIG. 15, a method 1500 includes controlling actuation of at least two spatially separated force applying elements on the torso support, each force applying element adapted to apply force to a localized region of the torso of the subject, as indicated at 1502. For example, method 1500 may include controlling actuation of the at least two spatially separated force applying elements based at least in part on a temporal pattern, as indicated at 1504, or controlling actuation of the at least two spatially separated force applying elements based at least in part on a spatial pattern, as indicated at 1506, or both. In an aspect, method 1500 includes controlling actuation of the at least two force applying elements according to a pre-defined pattern selectable from a plurality of pre-defined patterns, as indicated at 1508. Controlling actuation according to a pre-defined pattern may be generally as described in connection with FIG. 14, for example.

In further aspects, method 1500 includes controlling a thermal stimulator to deliver a thermal stimulus to at least a portion of the torso of the subject, at 1510, controlling a neural stimulator to deliver a neural stimulus to a neural structure in the torso of the subject, at 1512, and/or controlling a muscle stimulator to deliver a stimulus to activate a muscle in the torso of the subject, at 1514.

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture, limited to patentable subject matter under 35 §USC 101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof, limited to patentable subject matter under 35 U.S.C. §101. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, limited to patentable subject matter under 35 U.S.C. §101, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc) and so forth).

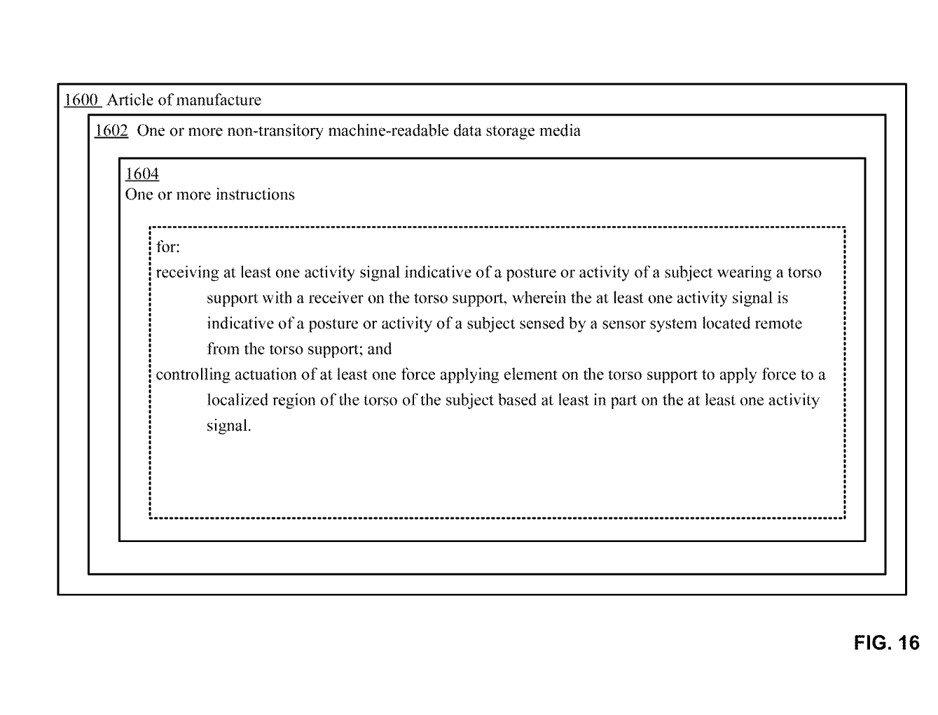

FIG. 16 depicts an article of manufacture 1600 that includes one or more non-transitory machine-readable data storage media 1602 bearing one or more instructions 1604 for receiving at least one activity signal indicative of a posture or activity of a subject wearing a torso support with a receiver on the torso support, wherein the at least one activity signal is indicative of a posture or activity of a subject sensed by a sensor system located remote from the torso support; and controlling actuation of at least one force applying element on the torso support to apply force to a localized region of the torso of the subject based at least in part on the at least one activity signal.

Instructions 1604 depicted in FIG. 16 correspond to the method 800 shown in FIG. 8. Other variants of methods as depicted in FIGS. 9-15 and as described herein can be implemented through the use of non-transitory machine-readable data storage media bearing one or more suitable instructions.

In an aspect, the one or more non-transitory machine-readable data storage media 1602 bear one or more instructions for carrying out the variants of method 900 as shown in FIG. 9, e.g., receiving the at least one activity signal from the sensor system, one or more instructions for receiving the at least one activity signal from a base station in communication with the sensor system, or one or more instructions for receiving the at least one activity signal from a network including one or more computing devices in communication with the sensor system, receiving a video signal, receiving a signal indicative of an image, receiving a signal indicative of pressure, receiving a signal indicative of motion, or receiving a signal indicative of proximity of the subject to a location, and/or additionally determining the proximity of the subject to the location based on the strength of the at least one activity signal.

In an aspect, the one or more non-transitory machine-readable data storage media 1602 bear one or more instructions for carrying out the variants of method 1000 as shown in FIG. 10, e.g., one or more instructions for determining the presence of the subject in a bed based on the at least one activity signal, wherein the one or more instructions for receiving the at least one activity signal include one or more instructions for receiving a signal indicative of the presence of the subject in a bed, optionally in combination with one or more instructions for determining preparation of the subject for sitting up on or rising from the bed based on the at least one activity signal, wherein the one or more instructions for receiving the at least one activity signal include one or more instructions for receiving a signal indicative of motion, force or pressure produced by the subject, and/or one or more instructions for determining the presence of the subject in a chair or seat based on the at least one activity signal, wherein the one or more instructions for receiving the at least one activity signal include one or more instructions for receiving a signal indicative of the presence of the subject in the chair or seat, optionally in combination with one or more instructions for determining preparation of the subject for leaning forward in or rising from the chair or seat, wherein the one or more instructions for receiving the at least one activity signal include one or more instructions for receiving a signal indicative of motion, force or pressure produced by the subject.

In an aspect, the one or more non-transitory machine-readable data storage media 1602 bear one or more instructions for carrying out the variants of method 1100 as shown in FIG. 11, e.g., one or more instructions for determining proximity of the subject to a bed based on the at least one activity signal, wherein the one or more instructions for receiving the at least one activity signal include one or more instructions for receiving a signal indicative of the proximity of the subject to a bed, optionally with one or more instructions for determining preparation of the subject for sitting or lying down on the bed, wherein the one or more instructions for receiving the at least one activity signal include one or more instructions for receiving a signal indicative of motion, force or pressure produced by the subject; one or more instructions for receiving at least one activity signal indicative of the proximity of the subject to a chair or seat, optionally with one or more instructions for receiving at least one activity signal indicative of motion, force or pressure produced by the subject, the motion, force or pressure indicative of preparation of the subject for rising from the chair or seat; and/or one or more instructions for determining crossing of a perimeter by the subject, wherein the one or more instructions for receiving the at least one activity signal include one or more instructions for receiving a signal indicative of crossing of the perimeter by the subject.

In an aspect, the one or more non-transitory machine-readable data storage media 1602 bear one or more instructions for carrying out the variants of method 1200 as shown in FIG. 12, e.g., one or more instructions for processing the at least one activity signal to determine the posture or activity of the subject, and one or more instructions for controlling actuation of the at least one force applying element based at least in part on the determined posture or activity of the subject.

In an aspect, the one or more non-transitory machine-readable data storage media 1602 bear one or more instructions for carrying out the variants of method 1300 as shown in FIG. 13, e.g., one or more instructions for receiving a processed activity signal that specifies the posture or activity of the subject from the at least one sensor system, wherein the processed activity signal has been processed by the at least one sensor system to specify a posture or activity of the subject, and one or more non-transitory machine-readable data storage media bear one or more instructions for controlling actuation of the at least one force applying element based at least in part on the posture or activity of the subject specified by the processed activity signal. Alternatively, or in addition, the one or more non-transitory machine-readable data storage media 1602 may bear one or more instructions for receiving a processed activity signal specifying at least one instruction corresponding to the posture or activity of the subject, or one or more instructions for receiving a signal indicative of an identity of the subject, as well as one or more instructions for controlling actuation of the at least one force applying element based at least in part on the identity of the subject. The one or more non-transitory machine-readable data storage media 1602 may bear one or more instructions for carrying out various addition aspects as depicted in FIG. 13, e.g. controlling actuation of the at least one force applying element to apply compressive force to the skin of the subject, controlling actuation of the at least one force applying element to apply tensile or shear force to the skin of the subject, controlling actuation of the at least one force applying element to control a stiffness of the at least one force applying element, controlling actuation of the at least one force applying element to control a dimension of the at least one force applying element, and controlling actuation of the at least one force applying element to control a position of the at least one force applying element.

In an aspect, the one or more non-transitory machine-readable data storage media 1602 bear one or more instructions for carrying out the variants of method 1400 as shown in FIG. 14, e.g., one or more instructions for controlling actuation of the at least one force applying element based at least in part on a temporal pattern, one or more instructions for controlling actuation of the at least one force applying element based at least in part on a spatial pattern, one or more instructions for controlling actuation of the at least one force applying element by controlling a pattern of force applied by the at least one force applying element, or one or more instructions for controlling actuation of the at least one force applying element by controlling a pattern of motion generated by the at least one force applying element. The one or more non-transitory machine-readable data storage media 1602 may bear one or more instructions for controlling actuation of the at least one force applying element according to a pre-defined pattern selected from a plurality of pre-defined patterns, possibly in combination with one or more instructions for receiving an input from a user input device and selecting the pre-defined pattern from the plurality of pre-defined patterns based upon the input, and/or one or more instructions for selecting the pre-defined pattern from the plurality of pre-defined patterns based at least in part upon the at least one activity signal. As discussed herein above, the plurality of pre-defined patterns includes patterns corresponding to a plurality of pre-defined postures or activities of the subject, e.g. standing, sitting, lying, walking, getting up, sitting down, or lying down.

In an aspect, the one or more non-transitory machine-readable data storage media 1602 bear one or more instructions for carrying out the variants of method 1500 as shown in FIG. 15, e.g., one or more instructions for controlling actuation of at least two spatially separated force applying elements on the torso support, each of the at least two spatially separated force applying elements adapted to apply force to a localized region of the torso of the subject. The one or more instructions for controlling actuation of the at least two spatially separated force applying elements on the torso support may include one or more instructions for controlling actuation of the at least two spatially separated force applying elements based at least in part on a temporal pattern, at least in part on a spatial pattern, or according to a pre-defined pattern selectable from a plurality of pre-defined patterns. In addition, the one or more non-transitory machine-readable data storage media may bear one or more instructions for controlling a thermal stimulator to deliver a thermal stimulus to at least a portion of the torso of the subject, one or more instructions for controlling a neural stimulator to deliver a neural stimulus to a neural structure in the torso of the subject, and/or one or more instructions for controlling a muscle stimulator to deliver a stimulus to activate a muscle in the torso of the subject.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.” With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A torso support system comprising:
a remote sensor system including:
at least one sensor adapted to detect an input indicative of a posture or activity of a subject; and
at least one transmitter adapted for transmitting at least one activity signal indicative of the posture or activity of the subject; and
a torso support including:
at least one force applying element adapted to apply force to a localized region of a torso of a subject;
at least one positioning element adapted to position the at least one force applying element with respect to the torso of the subject;
at least one receiver adapted to receive at least one activity signal indicative of the posture or activity of the subject detected by the at least one sensor system located remote from the torso support; and
control circuitry configured to control actuation of the at least one force applying element based at least in part on the at least one activity signal received by the at least one receiver.

2.-3. (canceled)

4. The torso support system of claim 1, wherein the at least one transmitter is adapted for transmitting at least one activity signal to a network including one or more computing devices and wherein the at least one receiver is adapted to receive at least one activity signal from the network including one or more computing devices.

5. The torso support system of claim 1, wherein the at least one transmitter is adapted for transmitting at least one activity signal to a base station and wherein the at least one receiver is adapted to receive at least one activity signal from the base station.

6.-54. (canceled)

55. A torso support comprising:
at least one force applying element adapted to apply force to a localized region of a torso of a subject;
at least one positioning element adapted to position the at least one force applying element with respect to the torso of the subject;
at least one receiver adapted to receive at least one activity signal indicative of a posture or activity of the subject detected by at least one sensor system located remote from the torso support; and
control circuitry configured to control actuation of the at least one force applying element based at least in part on the at least one activity signal.

56.-102. (canceled)

103. The torso support of claim 55, wherein the control circuitry is configured to control actuation of the at least one force applying element based at least in part on a temporal pattern.

104.-110. (canceled)

111. The torso support of claim 55, comprising at least two spatially separated force applying elements each adapted to apply force to a localized region of the torso of the subject, wherein the at least two spatially separated force applying elements are positioned at different positions with respect to the torso of the subject by the at least one positioning element.

112.-123. (canceled)

124. A method of controlling a torso support comprising:
receiving at least one activity signal indicative of a posture or activity of a subject wearing a torso support with a receiver on the torso support, wherein the at least one activity signal is indicative of a posture or activity of a subject sensed by a sensor system located remote from the torso support; and
controlling actuation of at least one force applying element on the torso support to apply force to a localized region of the torso of the subject based at least in part on the at least one activity signal.

125.-147. (canceled)

148. The method of claim 124, wherein receiving the at least one activity signal includes receiving at least one signal indicative of crossing of a perimeter by the subject.

149.-164. (canceled)

165. The method of claim 124, including controlling actuation of the at least one force applying element according to a pre-defined pattern selected from a plurality of pre-defined patterns.

166.-167. (canceled)

168. The method of claim 165, wherein the plurality of pre-defined patterns includes patterns corresponding to a plurality of pre-defined postures or activities of the subject.

169.-170. (canceled)

171. The method of claim 165, including controlling actuation of at least two spatially separated force applying elements on the torso support, each force applying element adapted to apply force to a localized region of the torso of the subject.

172.-173. (canceled)

174. The method of claim 171, including controlling actuation of the at least two force applying elements according to a pre-defined pattern selectable from a plurality of pre-defined patterns.

175.-178. (canceled)

179. An article of manufacture comprising:
one or more non-transitory machine-readable data storage media bearing one or more instructions for:
receiving at least one activity signal indicative of a posture or activity of a subject wearing a torso support with a receiver on the torso support, wherein the at least one activity signal is indicative of a posture or activity of a subject sensed by a sensor system located remote from the torso support; and
controlling actuation of at least one force applying element on the torso support to apply force to a localized region of the torso of the subject based at least in part on the at least one activity signal.

180.-225. (canceled)

226. The torso support system of claim 1, wherein the at least one transmitter is adapted for transmitting at least one activity signal to the at least one receiver and the at least one receiver is adapted to receive the at least one activity signal from the at least one transmitter.

227. The torso support system of claim 1 wherein the at least one sensor includes at least one of a camera, a pressure sensor, a motion sensor, a proximity sensor, a perimeter sensor, a micro-impulse radar sensor, an optical sensor, an electromagnetic sensor, and an acoustic sensor.

228. The torso support system of claim 1, wherein the at least one sensor is adapted for use in connection with at least one of a vehicle, an item of furniture, a bed, a chair, a floor, a door, a hinge, a latch, a door frame, an arm rest, a handle, and a stair; wherein the at least one sensor is adapted for placement in or on a support rail, handle, armrest or a handle; or wherein the at least one sensor is a component of at least one of a seat pad, a seat, a bed, a bed pad, and a floor mat.

229. The torso support system of claim 1, wherein the remote sensor system includes remote signal processing circuitry configured to process the input indicative of a posture or activity of a subject to produce an activity signal, the activity signal specifying the posture or activity of the subject or an instruction corresponding to the posture or activity of the subject wherein the at least one transmitter is adapted for transmitting the activity signal; wherein the at least one receiver is adapted to receive the at least one activity signal; and wherein the control circuitry is configured to control actuation of the at least one force applying element based at least in part on the posture or activity of the subject or the instruction corresponding to the posture or activity specified by the at least one activity signal.

230. The torso support of claim 55, further comprising at least one identity signal receiver adapted to receive a signal indicative of an identity of the subject, wherein the control circuitry is configured to control actuation of the at least one force applying element based at least in part on the identity of the subject.

231. The torso support of claim 55, wherein the at least one receiver is adapted to receive the at least one activity signal from the at least one sensor system, from a network including one or more computing devices in communication with the at least one sensor system, or from a base station in communication with the at least one sensor system.

232. The torso support of claim 55, wherein the at least one force applying element includes at least one of a spring, an elastic material, a viscoelastic material, an actuator, a mechanical linkage, a piezoelectric actuator, a thermally responsive element, an expandable element, an inflatable element, a screw, a pneumatic element, and a hydraulic element.

233. The torso support of claim 55, wherein the control circuitry is configured to control actuation of the at least one force applying element by at least one of controlling a pattern of force applied by the at least one force applying element and controlling a pattern of motion generated by the at least one force applying element.

234. The torso support of claim 55, further comprising at least one of a thermal stimulus source, a neural stimulator, and a muscle stimulator.

235. The method of claim 124, including receiving the at least one activity signal from the sensor system, a base station in communication with the sensor system, or a network including one or more computing devices in communication with the sensor system.

236. The method of claim 124, wherein receiving the at least one activity signal includes receiving at least one of a video signal, a signal indicative of an image, a signal indicative of pressure, a signal indicative of motion, a signal indicative of proximity of the subject to a location.

237. The method of claim 124, including determining the presence of the subject in a bed, chair, or seat based on the at least one activity signal.

238. The method of claim 237, including determining preparation of the subject for sitting up on or rising from the bed or leaning forward in or rising from the chair based on the at least one activity signal, wherein receiving at least one activity signal includes receiving a signal indicative of motion, force or pressure produced by the subject.

239. The method of claim 124, including determining proximity of the subject to a bed, chair or seat based on the at least one activity signal.

240. The method of claim 239, including determining preparation of the subject for sitting or lying down on the bed or rising from the chair or seat based on the at least one activity signal, wherein receiving the at least one activity signal includes receiving a signal indicative of motion, force or pressure produced by the subject.

241. The method of claim 124, including processing the at least one activity signal to determine the posture or activity of the subject and controlling actuation of the at least one force applying element based at least in part on the determined posture or activity of the subject.

242. The method of claim 124, including controlling actuation of the at least one force applying element based at least in part on a posture or activity of the subject specified by a processed activity signal, wherein receiving the at least one activity signal includes receiving the processed activity signal, and wherein the processed activity signal has been processed to specify the posture or activity of the subject.

243. The method of claim 124, including controlling actuation of the at least one force applying element based at least in part on at least one instruction corresponding to the posture or activity of the subject, wherein receiving the at least one activity signal includes receiving a processed activity signal, and wherein the processed activity signal has been processed to specify the at least one instruction.

244. The method of claim 124, including receiving a signal indicative of an identity of the subject and controlling actuation of the at least one force applying element based at least in part on the identity of the subject.

245. The method of claim 124, including controlling actuation of the at least one force applying element based at least in part on at least one of a temporal pattern and a spatial pattern.

246. The method of claim 165, including at least one of selecting the pre-defined pattern from the plurality of re-defined patterns based at least in part upon the at least one activity signal and receiving an input from a user input device and selecting the pre-defined pattern from the plurality of pre-defined patterns based upon the input.

247. The method of claim 171, including controlling actuation of the at least two spatially separated force applying elements based at least in part on at least one of a temporal pattern and a spatial pattern.
